(19) United States
(12) Patent Application Publication
(10) Pub. No.: US 2025/0236668 A1
(43) Pub. Date: Jul. 24, 2025

(54) ANTIBODIES AND METHODS OF MAKING AND USING SAME

(71) Applicant: ImaginAb, Inc., Inglewood, CA (US)

(72) Inventors: Alessandro Mascioni, Los Angeles, CA (US); Fang Jia, Cerritos, CA (US); Leticia Maria De Souza Cordeiro, Redondo Beach, CA (US); Kelley C. Atkinson, Anaheim, CA (US); Patrick Joyce, Gardena, CA (US); Argin Aivazian, Burbank, CA (US)

(21) Appl. No.: 18/881,583

(22) PCT Filed: Jul. 6, 2023
(86) PCT No.: PCT/US2023/069725

§ 371 Date: Jan. 6, 2025

Related U.S. Application Data
(63) Continuation of application No. PCT/US2022/073565, filed on Jul. 8, 2022.
(60) Provisional application No. 63/367,944, filed on Jul. 8, 2022.

(51) Int. Cl. C07K 16/28; A61K 39/00; C07K 16/40

(52) U.S. Cl. CPC C07K 16/28; C07K 16/40; C12Y 401/01; A61K 2039/505; C07K 2317/24; C07K 2317/626; C07K 2317/94

(57) ABSTRACT

Antibodies having enhanced biodistribution and/or pharmacokinetics obtained through disruption of one or more clusters of surface-exposed positively charged amino acids are provided. Also provided are methods of enhancing the biodistribution and/or pharmacokinetics of an antibody. The enhanced antibodies find use in immunotherapy, radiotherapy, and in vivo imaging.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING

The present application is being filed along with a Sequence Listing in electronic format. The Sequence Listing is provided as a file entitled IGNAB063WO_SEQLIST.xml, which was created on Jul. 2, 2023, which is 66,818 bytes in size. The information in the electronic Sequence Listing is hereby incorporated by reference in its entirety.

### BACKGROUND

### Field

The present disclosure generally relates to enhanced biodistribution and/or pharmacokinetics of antibodies (including minibodies and cys-diabodies), and compositions therefor.

### Description of the Related Art

The present disclosure generally relates to antibodies, including antigen binding fragments thereof, such as minibodies and cys-diabodies. Antibodies can be used to bind targets for therapeutic or diagnostic uses.

### SUMMARY

Provided herein is a variant antibody comprising at least one disrupted cluster of surface-exposed positively charged amino acids, wherein the variant antibody varies from an original antibody comprising an original cluster comprising at least two surface-exposed, positively charged amino acids within about 30 angstroms of each other by having a substitution of at least one of the surface-exposed positively charged amino acids of the original cluster with a negatively charged or non-charged amino acid.

Also provided is an antibody comprising a variant polypeptide that varies from an original polypeptide comprising an original sequence X₁X₂X₃X₄X₅X₆X₇ (SEQ ID NO: 1), wherein the original sequence is outside of any CDR of the original polypeptide, wherein X₁, X₂, X₃, X₄, X₅, X₆, and X₇ are each independently any amino acid with the proviso that at least two are surface-exposed, positively charged amino acids that form a cluster of surface-exposed positively charged amino acids in the original polypeptide, wherein the variant polypeptide varies from the original polypeptide by having a modification of at least one of the at least two surface exposed, positively charged amino acids of the original sequence that reduces the positive charge (e.g., reduces at least 33% of positive charges) attributed to the original sequence.

Further provided herein is a variant antibody comprising at least one disrupted cluster of positively charged amino acids, wherein the variant antibody varies from an original antibody comprising an original cluster comprising at least two positively charged amino acids within 12 residues of each other by having a substitution of at least one of the positively charged amino acids of the original cluster with a negatively charged or non-charged amino acid, wherein the original cluster is outside of any CDR of the original antibody.

Also provided is a minibody or cys-diabody comprising a variant polypeptide that varies from an original polypeptide comprising a light chain variable region (V_{L}) FR2 comprising an original sequence X₁X₂X₃X₄X₅X₆X₇ (SEQ ID NO:1), wherein X₁, X₂, X₃, X₄, X₅, X₆, and X₇ are each independently any amino acid with the proviso that at least two are surface-exposed, positively charged amino acids that form a cluster of surface-exposed positively charged amino acids in the original polypeptide, wherein the variant polypeptide varies from the original polypeptide by having a modification of at least one of the at least two surface exposed, positively charged amino acids of the original sequence that reduces at least 33% of positive charges attributed to the original sequence.

Provided herein is a minibody or cys-diabody comprising a light chain variable region (V_{L}) comprising a FR2 sequence comprising X₁X₂X₃QAX₆X₇, (SEQ ID NO:4), wherein X₁ is a surface-exposed, positively charged amino acid, wherein X₂, X₃, and X₆, are each independently any negatively charged or non-charged amino acid, and wherein X₇ is either glutamine or lysine.

Provided herein is a minibody or cys-diabody comprising a light chain variable region (V_{L}) comprising (by IMGT numbering): A49 and either: (i) Q51; or (ii) Q48 and K51.

Also provided is a minibody comprising an upper hinge sequence of EPGSSDGTHT (SEQ ID NO:39).

Provided herein is a composition comprising: any one of the antibodies (e.g., variant antibodies), minibodies or cys-diabodies of the present disclosure; and a pharmaceutically acceptable carrier.

Also provided is a nucleic acid encoding the variant polypeptide of any one of the antibodies (e.g., variant antibodies) herein, or any one of the variant antibodies herein, or any one of the minibodies or cys-diabodies of the present disclosure.

Also provided is a genetically engineered host cell comprising any one of the nucleic acids of the present disclosure, or a combination thereof.

Provided herein is a method of enhancing biodistribution and/or pharmacokinetics of an antibody, comprising: identifying an original antibody comprising a polypeptide comprising at least one cluster of surface-exposed positively charged amino acids, the cluster comprising at least two surface-exposed, positively charged amino acids within 30 angstroms of each other; substituting at least one of the at least two surface-exposed, positively charged amino acids of the at least one cluster with a negatively charged or non-charged amino acid to thereby disrupt the at least one cluster, whereby a variant antibody having enhanced biodistribution and/or pharmacokinetics compared to the original antibody is generated.

Also provided is a method of enhancing biodistribution and/or pharmacokinetics of an antibody, comprising: identifying an original antibody comprising a polypeptide comprising a cluster of at least two surface-exposed, positively charged amino acids within 12 residues of each other; and substituting at least one of the at least two surface-exposed, positively charged amino acids of the cluster with a negatively charged or non-charged amino acid to disrupt the cluster, thereby generating a variant antibody having enhanced biodistribution and/or pharmacokinetics compared to the original antibody.

Provided herein is a method of making a labeled antibody, comprising: selecting a germline sequence for a light chain variable region (V_{L}) of an antibody, wherein the germline sequence comprises no cluster of at least two positively charged amino acids within 3 residues of each other in a framework region 2 (FR2) of the germline sequence; isolating one or more target-specific antibodies among a population of antibodies comprising a V_{L} derived from the germline sequence and having variations in the germline sequence across the population, wherein the one or more target-specific antibodies comprises no cluster of at least two positively charged amino acid within 3 residues of each other in a V_{L} FR2 sequence; and labeling the one or more target-specific antibodies.

Also provided is an antibody made by any one of the methods of the present disclosure.

Provided herein is a method of treating a subject, comprising: identifying a subject in need of treatment with any one of the antibody (e.g., variant antibody), minibody, or cys-diabody of the present disclosure; and administering to the subject a therapeutically effective amount of the antibody or minibody, or a composition comprising same.

Further provided is a method of treating a subject for a cancer, comprising: identifying a subject in need of treatment for a cancer; and administering to the subject a therapeutically effective amount of any one of the antibody (e.g., variant antibody), minibody, or cys-diabody of the present disclosure, a composition comprising same, to thereby treat the cancer.

Provided herein is a method of radiotherapy, comprising: identifying a subject in need of radiotherapy; and administering to the subject a therapeutically effective amount of any one of the antibody (e.g., variant antibody), minibody, or cys-diabody of the present disclosure, a composition comprising same, wherein the antibody, minibody, or cys-diabody comprises a radionuclide.

Also provided is a method of imaging a subject, comprising: administering to a subject a composition comprising an effective amount of any one of the antibody (e.g., variant antibody), minibody, or cys-diabody of the present disclosure, a composition comprising same, wherein the antibody, minibody, or cys-diabody is detectably labeled; and imaging the subject to detect the labeled antibody, minibody, or cys-diabody in the subject.

### BRIEF DESCRIPTION OF THE DRAWINGS

FIG. 1 is a block diagram showing a non-limiting example of a method of enhancing biodistribution and/or pharmacokinetics of an antibody.

FIG. 2 is a block diagram showing a non-limiting example of a method of enhancing biodistribution and/or pharmacokinetics of an antibody.

FIG. 3 is a block diagram showing a non-limiting example of a method of making a labeled antibody.

FIG. 4 is a block diagram showing a non-limiting example of a method of treating a subject.

FIGS. 5A-5C are a collection of non-limiting examples of amino acid sequences of minibodies.

FIG. 6 is a graph showing a comparison of biodistribution between original and variant minibodies, according to some non-limiting embodiments of the present disclosure.

FIGS. 7A-7D are a collection of non-limiting examples of amino acid sequences of antibody fragments.

FIG. 8 is a graph showing a comparison of biodistribution between original and variant minibodies, according to some non-limiting embodiments of the present disclosure.

FIG. 9 is a graph showing a comparison of biodistribution between original and variant minibodies, according to some non-limiting embodiments of the present disclosure.

FIG. 10 is a schematic diagram of some non-limiting structural features of a minibody.

FIG. 11 is a schematic diagram of some non-limiting structural features of a cys-diabody.

FIG. 12 is a collection of non-limiting examples of C_{H}3 amino acid sequences.

### DETAILED DESCRIPTION

The biodistribution and pharmacokinetics of antibodies, minibodies and cys-diabodies, when administered to a subject, is an important determinant of their safety and efficacy. Improved biodistribution and pharmacokinetics can affect the dosing and schedule of administration of antibody therapeutics, such as during radioimmunotherapy or when used as antibody-drug conjugates. It can also affect the dosing and imaging time point for radiodiagnostic imaging with antibodies, minibodies and cys-diabodies. The biodistribution and pharmacokinetics of antibodies can vary depending on a number of factors, including some properties of surface-exposed electrostatic charges of the antibody.

Provided herein are antibodies (including minibodies and cys-diabodies) having enhanced biodistribution and/or pharmacokinetics and methods for enhancing the biodistribution and/or pharmacokinetics of antibodies. The antibodies (e.g., variant antibodies) of the present disclosure generally have a disrupted cluster of positively charged amino acids compared to an original antibody, and can have enhanced biodistribution and/or pharmacokinetics compared to the original antibody having the cluster and on which the antibody having the disrupted cluster is based.

Without being bound by theory, it is thought that the cluster of positively charged amino acids form a patch of positive electrostatic charges as described by an isopotential surface over the antibody, and this localized charge concentration may contribute to accumulation of the antibody in radiosensitive or drug sensitive tissues, such as the kidneys, when administered to a subject. Undesired antibody accumulation in the kidneys can be due to increased capture at the kidney glomeruli or proximal tubules and retention (and/or increased reabsorption and retention at these renal sub-compartments) relative to a variant antibody as provided herein.

Disruption of the cluster of positively charged amino acids, as provided herein, can reduce the accumulation of the antibody in the kidney. In some embodiments, disruption of the cluster reduces the accumulation of the antibody in the kidney by at least about 10%, 20%, 30%, 40%, 50%, 60%, 70%, 80%, or more, or by a percentage in a range defined by any two of the preceding values (e.g., 10-80%, 10-50%, 30-80%, 40-70%, etc.) In some embodiments, disruption of the cluster increases accumulation of the antibody in the liver. In some embodiments, the change in biodistribution due to disruption of the cluster is readily observed for an antibody construct having a molecular weight (as a dimer) of ˜50-80 kDa, such as a minibody or a cys-diabody. In general, modification of the original antibody by disrupting the cluster of positively charged amino acids at least maintains the same binding specificity and binding affinity of the original antibody, while enhancing the biodistribution and/or pharmacokinetics. In some embodiments, the antibody (or antigen-binding fragment thereof) has a molecular weight of 15-160 kDa. In some embodiments, the antibody (or antigen-binding fragment thereof) has a molecular weight of 15-110 kDa. In some embodiments, the antibody (or antigen-binding fragment thereof) has a molecular weight of about 50-80 kDa.

In some embodiments, the biodistribution of the antibody can be controlled by modifying the electrostatic charge associated with the cluster. As noted above, in some embodiments, the cluster of positively charged amino acids can promote accumulation in the kidneys, and reducing the positive charge, or increasing the negative charge associated with the cluster can promote accumulation away from the kidneys, e.g., promote accumulation in the liver. Thus, in some embodiments, biodistribution of a cytotoxic agent (e.g., toxin) or radionuclide conjugated to the antibody can be biased toward the kidneys by increasing the positive charges associated with the cluster, and biodistribution of a cytotoxic agent (e.g., toxin) or radionuclide conjugated to the antibody can be biased toward the liver by reducing the positive charges, or increasing the negative charges associated with the cluster. In some embodiments, distribution of the antibody to the liver is promoted where a liver disorder (e.g., liver cancer) is treated by administering the antibody.

In the radioimmunotherapy context, the accumulation of the antibody in the kidney can lead to renal toxicity. Thus, the antibodies of the present disclosure having reduced accumulation in the kidney can allow for administering a greater dose of a radiolabeled antibody (e.g., greater specific activity, greater frequency and/or number of administration) while reducing renal toxicity.

### Terms

All terms have their ordinary and customary meaning as understood by one of ordinary skill in the art, in view of the present disclosure.

The term “antibody” as used herein includes all varieties of antibodies, including antigen binding fragments thereof. Further included are constructs that include 1, 2, 3, 4, 5, and/or 6 CDRs. In some embodiments, tandem scFvs can be provided, which can provide two arms with bivalent binding. In some embodiments, these CDRs can be distributed between their appropriate framework regions in a traditional antibody. In some embodiments, the CDRs can be contained within a heavy and/or light chain variable region. In some embodiments, the CDRs can be within a heavy chain and/or a light chain. In some embodiments, the CDRs can be within a single peptide chain. Unless otherwise denoted herein, the antibodies described herein bind to the noted target molecule. The term “target” or “target molecule” denotes the protein to which the antigen binding construct binds at some level of specificity under physiological conditions.

The term “antibody” includes, but is not limited to, genetically engineered or otherwise modified forms of immunoglobulins, such as intrabodies, chimeric antibodies, fully human antibodies, humanized antibodies, antibody fragments, single-chain variable fragment (scFv), and heteroconjugate antibodies (for example, bispecific antibodies, diabodies, triabodies, tetrabodies, etc.). Also, the term “antibody” includes camelid derived immunoglobulins like single heavy-chain antibodies and nanobodies. “Antibody fragment” includes, without limitation, Fab′, F(ab′)₂, Fab, Fv, rIgG (reduced IgG), scFv fragments, scFv-Fc, single domain fragments (e.g., nanobodies), peptibodies, Nanobodies®, Nanobody®-Fc, minibodies, and diabodies. The term “antibody” includes scFv and minibodies. Thus, each and every embodiment provided herein in regard to “antibodies” is also envisioned as scFv and/or minibody embodiments, unless explicitly denoted otherwise. The term “antibody” includes a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. In some embodiments, a full-length antibody can be composed of two identical pairs of polypeptide chains, each pair having one “light” and one “heavy” chain (connected through a disulfide bond). The recognized immunoglobulin genes include the kappa, lambda, alpha, gamma, delta, epsilon, hinge, and mu constant region genes, as well as the myriad immunoglobulin variable region genes. For full length chains, the light chains are classified as either kappa or lambda. For full length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of up to about 149 or more amino acids primarily responsible for antigen recognition. The terms variable light chain (V_{L}) and variable heavy chain (V_{H}) refer to these regions of light and heavy chains respectively. As used in this application, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (for example, F(ab′)₂) with the same binding specificity. In some embodiments, the antibody binds specifically to a desired target.

“Antibody” may also include one or more immunoglobulin chains that are chemically conjugated to, or expressed as, fusion proteins with other proteins. It also includes bispecific antibodies. A bispecific or bifunctional antibody is an artificial hybrid antibody having two different heavy/light chain pairs and two different binding sites.

As used herein “antibody” can also mean other antigen-binding fragments or antibody portions of the present disclosure including, bispecific scFv antibodies where the antibody molecule recognizes two different epitopes, single binding domains (sdAb or Nanobodies®), minibodies, and cys-diabodies.

The term “antibody fragment” includes, but is not limited to, one or more antigen binding fragments of antibodies alone or in combination with other molecules, including, but not limited to Fab′, F(ab′)₂, Fab, Fv, rIgG (reduced IgG), scFv fragments, scFv-Fc, single domain fragments (Nanobodies®), peptibodies, Nanobodies®, Nanobody®-Fc, minibodies, diabodies and cys-diabodies. The term “scFv” refers to a single chain Fv (“fragment variable”) antibody in which the variable domains of the heavy chain and of the light chain of a traditional two chain antibody have been joined to form one chain.

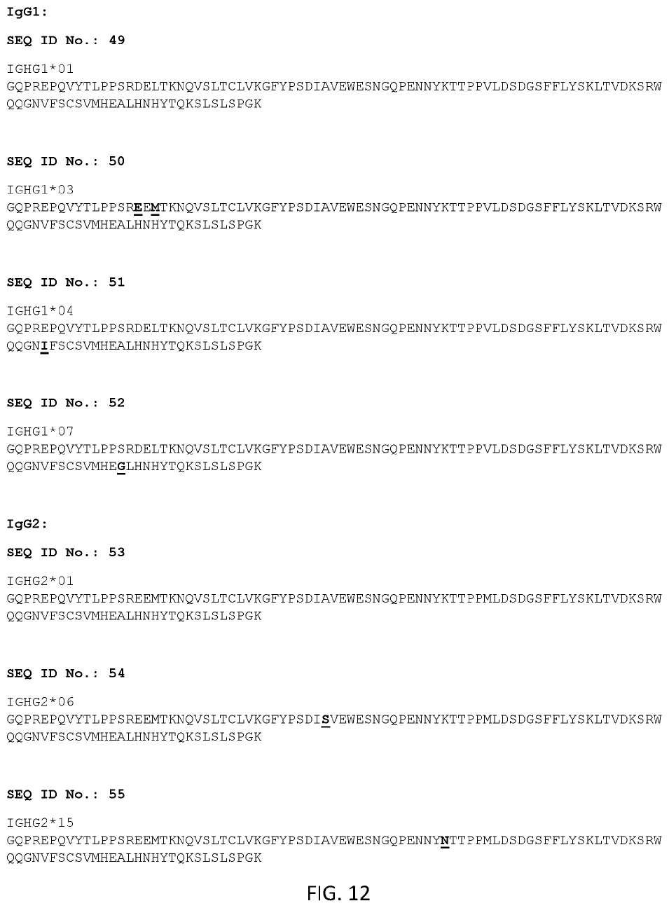

A minibody is an antibody format that has a smaller molecular weight than the full-length antibody while maintaining the bivalent binding property against an antigen. Because of its smaller size (˜70-90 kDa, preferably ˜80 kDa), absence of C_{H}2 domain that binds Fc-gamma and FcRn receptors, and absence of glycosylation, the minibody has a faster clearance from the blood system and potentially enhanced penetration when targeting tumor tissue. With the ability for strong targeting combined with rapid clearance relative to full size antibodies, the minibody is advantageous for diagnostic and therapeutic applications and delivery of radioactive payloads for which prolonged circulation times may result in adverse patient dosing or dosimetry. In some embodiments, it can also be advantageous for delivery of a cytotoxic payload (such as in the case of antibody drug conjugates, ADC) due to the above-mentioned features such as tumor penetration and faster clearance. A “minibody” as described herein, encompasses a homodimer, wherein each monomer is a single-chain variable fragment (scFv) linked to a human IgG C_{H}3 domain by a hinge sequence. A non-limiting, schematic diagram of a minibody is shown in FIG. 10. In some embodiments, a minibody is a bivalent or bispecific, covalently bound heterodimer of ˜80 kDa. In some embodiments, each monomer (half-molecule) is comprised of a variable heavy (V_{H}) domain linked to the corresponding variable light (V_{L}) domain by an approximate 15-18 amino acid Gly-Ser-rich linker sequence. In some embodiments, each single-chain variable fragment (scFv) is linked to a human IgG1, IgG2, IgG3 or IgG4 C_{H}3 domain by a hinge sequence.

The term “hinge” denotes at least a part of a hinge region for an antigen binding construct, such as an antibody or a minibody, a scFv-Fc, or a Nanobody®-Fc. A hinge region can include a combination of the upper hinge, core (or middle) hinge and lower hinge regions. In some embodiments, the hinge is defined according to any of the antibody hinge definitions. Native IgG1, IgG2, and IgG4 antibodies have hinge regions having of 12-15 amino acids. IgG3 has an extended hinge region, having 62 amino acids, including 21 prolines and 11 cysteines. The functional hinge region of naturally occurring antibodies, deduced from crystallographic studies, can extend from amino acid residues 216-237 of the IgG1 H chain (EU numbering) and include a small segment of the N terminus of the C_{H}2 domain in the lower hinge, with the lower hinge being the N terminus of C_{H}2 domain. The hinge can be divided into three regions; the “upper hinge,” the “core,” and the “lower hinge”.

The term “upper hinge” denotes the first part of the hinge that starts at the end of the scFv. The upper hinge includes the amino acids from the end of the scFv up to, but not including, the first cysteine residue in the core hinge. The term “effective upper hinge” denotes that enough of the sequence is present to allow the section to function as an upper hinge; the term encompasses functional variants and fragments of the designated hinge section.

The term “core hinge” denotes the second part of the hinge region that is C-terminal to the upper hinge. The core hinge can contain the inter-chain disulfide bridges and a high content of prolines.

The term “lower hinge” denotes the third part of the hinge region that is C-terminal to the core hinge. In the context of a minibody or antibody fragment, the lower hinge connects to the C_{H}3 domain Mb. As above, the term “effective lower hinge” denotes that enough of the sequence is present to allow the section to function as a lower hinge; the term encompasses functional variants and fragments of the designated hinge section. The term “lower hinge” as used herein can encompass various amino acid sequences including naturally occurring IgG lower hinge sequences and artificial extension sequences in place of one another or a combination thereof provided herein. In some embodiments, the various extensions can be considered to be a lower hinge region in its entirety or a replacement.

In some embodiments a lower hinge can be a native IgG1, 2, 3 or 4 lower hinge, and/or a Gly-Ser sequence (G₃)Sₙ or (G₄)Sₙ(n can be any number of S's; in some embodiments it is 1 or 2) and/or no lower hinge and/or any combination of amino acids (doesn't have to be G's and S's). In some embodiments, the lower hinge can comprise GGGSSGGGSG (SEQ ID NO:40).

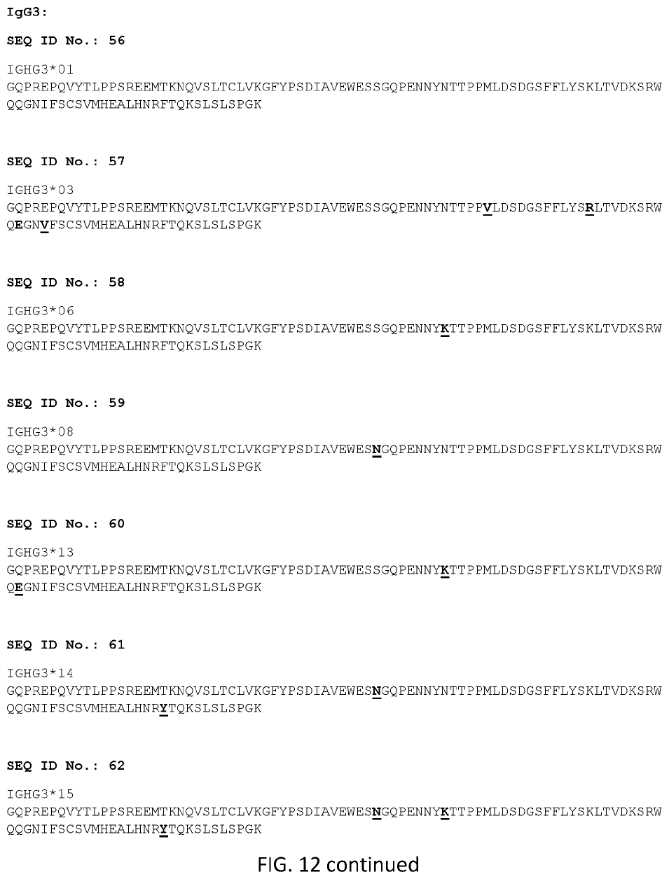

The term “diabody” denotes a dimer that comprises heavy chain (V_{H}) domains and light-chain variable (V_{L}) domains. Each heavy chain domain is connected to a light chain domain through a linker, forming a monomer. Two monomers are covalently linked through a bridging moiety to form the diabody. A “cys-diabody” denotes a diabody whose monomer chains are covalently linked by a disulfide bond. A non-limiting, schematic diagram of a cys-diabody is shown in FIG. 11. A cys-diabody can have a molecular weight of ˜50 kDa.

The term “extension sequence” (e.g., in a diabody context) denotes a region that connects a first V_{H} domain to a second V_{H} domain or a first V_{L} to a second V_{L} domain, in for example, a diabody. Extension sequences can connect the domains through the C-terminus of each domain. In some embodiments, extension sequences connect the domains through covalent bonds. In some embodiments, the extension sequence will include one or more cysteine, allowing for one or more disulfide bonds to be formed between two such extension sequences. An example of a pair of extension sequences is shown in the schematic diagrams on the right side of FIG. 11, as the line with two cysteines connecting either the two heavy chain domains or the two light chain domains. A non-limiting example of an extension sequence of -(Gly)₂-(Cys) is shown in the schematic diagrams on the left side of FIG. 11. In some embodiments, the extension sequence includes 1, 2, 3, or more cysteines per monomer chain. While the extension sequence will be towards the C-terminus of the constructs in FIG. 11, it need not be the absolute last amino acid in the variable domain. That is, the extension sequence can be positioned slightly N-terminal to the C-terminus. For example, the extension sequence can be placed within the 10 amino acids of the C-terminus of the monomer. Similarly, additional sequence can be placed between the native C-terminus and where the extension sequence starts. The extension sequence can connect V_{H} to V_{H} or V_{L} to V_{L} through a disulfide bond. In some embodiments, the extension sequence includes GGCPPCPPC (SEQ ID NO:72).

In some embodiments, a linker can be any suitable linker for the antibody (e.g., minibody, cys-diabody). In some embodiments, a linker sequence can include a motif that is (G₃)Sₙ or (G₄)Sₙ (n can be any integer; in some embodiments it is 1 or 2). In some embodiments, the linker can comprise GSTSGGGSGGGSGGGGSS (SEQ ID NO:41).

The term “treating” or “treatment” of a condition can refer to preventing the condition, slowing the onset and/or rate of development of the condition, reducing the risk of developing the condition, preventing and/or delaying the development of symptoms associated with the condition, reducing or ending symptoms associated with the condition, generating a complete or partial regression of the condition, or some combination thereof. The term “prevent” does not require the absolute prohibition of the disorder or disease.

A “therapeutically effective amount” or a “therapeutically effective dose” is an amount that produces a desired therapeutic effect in a subject, such as preventing, treating a target condition, delaying the onset of the disorder and/or symptoms, and/or alleviating symptoms associated with the condition. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and/or the route of administration. One skilled in the clinical and pharmacological arts will be able to determine a therapeutically effective amount through routine experimentation, for example by monitoring a subject's response to administration of a compound and adjusting the dosage accordingly, given the present disclosure. For additional guidance, see Remington: The Science and Practice of Pharmacy 21^{st} Edition, Univ. of Sciences in Philadelphia (USIP), Lippincott Williams & Wilkins, Philadelphia, PA, 2005.

The term “complementarity-determining domains” or “complementarity-determining regions (“CDRs”) interchangeably refer to the hypervariable regions of V_{L} and V_{H}. The CDRs are the target molecule-binding site of the antibody chains that harbors specificity for such target molecule. In some embodiments, there are three CDRs (CDR1-3, numbered sequentially from the N-terminus) in each V_{L} and/or V_{H}, constituting about 15-20% of the variable domains. The CDRs are structurally complementary to the epitope of the target molecule and are thus directly responsible for the binding specificity. The remaining stretches of the V_{L} or V_{H}, the so-called framework regions (FRs), exhibit less variation in amino acid sequence (Kuby, Immunology, 4th ed., Chapter 4. W.H. Freeman & Co., New York, 2000).

The positions of the CDRs and framework regions can be determined using various well-known definitions in the art, e.g., Kabat (Wu, T. T., E. A. Kabat. 1970. An analysis of the sequences of the variable regions of Bence Jones proteins and myeloma light chains and their implications for antibody complementarity. J. Exp. Med. 132: 211-250; Kabat, E. A., Wu, T. T., Perry, H., Gottesman, K., and Foeller, C. (1991) Sequences of Proteins of Immunological Interest, 5th ed., NIH Publication No. 91-3242, Bethesda, MD); Chothia (Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)); ImMunoGeneTics database (IMGT) (on the worldwide web at imgt.org/) Giudicelli, V., Duroux, P., Ginestoux, C., Folch, G., Jabado-Michaloud, J., Chaume, D. and Lefranc, M.-P. IMGT/LIGM-DB, the IMGT® comprehensive database of immunoglobulin and T cell receptor nucleotide sequences Nucl. Acids Res., 34, D781-D784 (2006), PMID: 16381979; Lefranc, M.-P., Pommi6, C., Ruiz, M., Giudicelli, V., Foulquier, E., Truong, L., Thouvenin-Contet, V. and Lefranc, G., IMGT unique numbering for immunoglobulin and T cell receptor variable domains and Ig superfamily V-like domains Dev. Comp. Immunol., 27, 55-77 (2003). PMID: 12477501; Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008); AbM (Martin et al., Proc. Natl. Acad. Sci. USA, 86:9268-9272 (1989); North (North B., Lehmann A., Dunbrack R. L., A new clustering of antibody CDR loop conformations, J. Mol. Biol. (2011) 406(2): 228-256); AHo (Honegger A., Pluckthun, Yet another numbering scheme for immunoglobulin variable domains: an automatic modeling and analysis tool, J. Mol. Biol. (2001) 309, 657-670); the contact definition (MacCallum et al., J. Mol. Biol., 262:732-745 (1996)), and/or the automatic modeling and analysis tool Honegger A, Pluckthun A. (world wide web at bioc dot uzh dot ch/antibody/Numbering/index dot html). In some embodiments, the framework regions are defined by the AHo numbering method as follows: FR1, 1-24; FR2, 41-57; FR3, 78-108, and FR4, 138-149.

An “antibody variable light chain” or an “antibody variable heavy chain” as used herein refers to a polypeptide comprising the V_{L} or V_{H}, respectively. The endogenous V_{L} is encoded by the gene segments V (variable) and J (junctional), and the endogenous V_{H} by V, D (diversity), and J. Each of V_{L} or V_{H} includes the CDRs as well as the framework regions. In this application, antibody variable light chains and/or antibody variable heavy chains may, from time to time, be collectively referred to as “antibody chains.” These terms encompass antibody chains containing mutations that do not disrupt the basic structure of V_{L} or V_{H}, as one skilled in the art will readily recognize. In some embodiments, full length heavy and/or light chains are contemplated. In some embodiments, only the variable region of the heavy and/or light chains are contemplated as being present.

Antibodies can exist as intact immunoglobulins or as a number of fragments produced by digestion with various peptidases. Thus, for example, pepsin digests an antibody below the disulfide linkages in the hinge region to produce F(ab)′₂, a dimer of Fab′ which itself is a light chain (V_{L}-C_{L}) joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is a Fab with part of the hinge region. (Paul, W. E., “Fundamental Immunology,” 3d Ed., New York: Raven Press, 1993). While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (for example, single chain Fv) or those identified using phage display libraries (see, for example, McCafferty, J. et al., “Phage antibodies: filamentous phage displaying antibody variable domains,” Nature, Vol. 348, No. 66301, pp. 552-554, 1990).

For preparation of monoclonal or polyclonal antibodies, any technique known in the art can be used (see, for example, Kohler, G. et al., “Continuous cultures of fused cells secreting antibody of predefined specificity,” Nature, Vol. 256, No. 5517, pp. 495-497, 1975; Kozbor, D. et al., “The production of monoclonal antibodies from human lymphocytes,” Immunology Today, Vol. 4, No. 3, pp. 72-79, 1983; Cole, et al., “Monoclonal Antibodies and Cancer Therapy,” Alan R. Liss, Inc., pp. 77-96, 1985; Wang, S., “Advances in the production of human monoclonal antibodies,” Antibody Technology Journal, Vol. 1, pp. 1-4, 2011; Sharon, J. et al., “Recombinant polyclonal antibodies for cancer therapy,” J. Cell Biochem., Vol. 96, No. 2, pp. 305-313, 2005; Haurum, J. S., “Recombinant polyclonal antibodies: the next generation of antibody therapeutics?,” Drug Discov. Today, Vol. 11, No. 13-14, pp. 655-660, 2006). Techniques for the production of single chain antibodies (U.S. Pat. No. 4,946,778) can be adapted to produce antibodies to polypeptides of this invention. Also, transgenic mice, or other organisms such as other mammals, may be used to express fully human monoclonal antibodies. Alternatively, phage display technology can be used to identify high affinity binders to selected antigens (see, for example, McCafferty et al., supra; Marks, J. D. et al., “By-passing immunization: building high affinity human antibodies by chain shuffling,” Biotechnology (N. Y.), Vol. 10, No. 7, pp. 779-783, 1992).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. In some embodiments, the terms “donor” and “acceptor” sequences can be employed. Humanization can be essentially performed following the method of Winter and co-workers (see, for example, Jones, P. T. et al., “Replacing the complementarity-determining regions in a human antibody with those from a mouse,” Nature, Vol. 321, No. 6069, pp. 522-525, 1986; Riechmann, L. et al., “Reshaping human antibodies for therapy,” Nature, Vol. 332, No. 6162, pp. 323-327, 1988; Verhoeyen, M. et al., “Reshaping human antibodies: grafting an antilysozyme activity,” Science, Vol. 239, No. 4847, pp. 1534-1536, 1988; Presta, L. G., “Antibody engineering,”, Curr. Op. Struct. Biol., Vol. 2, No. 4, pp. 593-596, 1992), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementarity determining region (“CDR”) residues and possibly some framework (“FR”) residues are substituted by residues from analogous sites in rodent antibodies.

The term “Fe region” or “Fc domain” or “Fc” denotes a C-terminal region of an immunoglobulin heavy chain. The “Fe region” may be a native sequence Fe region or a variant Fc region (e.g., a variant having one or more mutations that reduces an effector function, e.g., FcγR binding, and/or binding to the Fc neonatal receptor (FcRn), etc.). The Fc region of an immunoglobulin (e.g., IgG) generally comprises two constant domains, C_{H}2 and C_{H}3. The Fc region may or may not include a hinge region or sequence, as described herein. An Fc region can be present in dimer or monomeric form. In some embodiments, the Fe region is a human Fc region, or a variant thereof.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, for example, an enzyme, toxin, hormone, growth factor, and drug; or (b) the variable region, or a portion thereof, is altered, replaced or exchanged with a variable region having a different or altered antigen specificity.

A pharmaceutically acceptable carrier may be a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or some combination thereof. Each component of the carrier is “pharmaceutically acceptable” in that it is compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. The pharmaceutical compositions described herein may be administered by any suitable route of administration. A route of administration may refer to any administration pathway known in the art, including but not limited to aerosol, enteral, nasal, ophthalmic, oral, parenteral, rectal, transdermal (for example, topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intracranial, intrauterine, intravenous, subarachnoid, subcapsular, sublingual, subcutaneous, transmucosal, or transtracheal. In some embodiments, the antigen binding construct can be delivered intraoperatively as a local administration during an intervention or resection.

The phrase “specifically (or selectively) bind,” when used in the context of describing the interaction between an antigen, for example, a protein, to an antibody or antibody-derived binding agent, refers to a binding reaction that is determinative of the presence of the antigen in a heterogeneous population of proteins and other biologics, for example, in a biological sample, for example, a blood, serum, plasma or tissue sample. Thus, under designated immunoassay conditions, in some embodiments, the antibodies or binding agents with a particular binding specificity bind to a particular antigen at least two times the background and do not substantially bind in a significant amount to other antigens present in the sample. Specific binding to an antibody or binding agent under such conditions may require the antibody or agent to have been selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, for example, Harlow, E. & Lane D., “Using Antibodies, A Laboratory Manual,” Cold Spring Harbor Laboratory Press, 1998, for a description of immunoassay formats and conditions that can be used to determine specific immunoreactivity). Typically a specific or selective binding reaction will produce a signal at least twice over the background signal and more typically at least than 10 to 100 times over the background.

The term “equilibrium dissociation constant (K_{D}, M)” refers to the dissociation rate constant (k_{d}, time⁻¹) divided by the association rate constant (kₐ, time⁻¹ M⁻¹). Equilibrium dissociation constants can be measured using any known method in the art. The antibodies of the present invention generally will have an equilibrium dissociation constant of less than about 10⁻⁷ or 10⁻⁸ M, for example, less than about 10⁻⁹ M or 10⁻¹⁰ M, in some embodiments, less than about 10⁻¹¹ M, 10⁻¹² M, or 10⁻¹³ M.

The term “isolated,” when applied to a nucleic acid or protein, denotes that the nucleic acid or protein is essentially free of other cellular components with which it is associated in the natural state. In some embodiments, it can be in either a dry or aqueous solution. Purity and homogeneity can be determined using analytical chemistry techniques such as polyacrylamide gel electrophoresis or high-performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel or size exclusion chromatography analysis. In some embodiments, this can denote that the nucleic acid or protein is at least 85% pure, more preferably at least 95% pure, and most preferably at least 99% pure of molecules that are present under in vivo conditions.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (for example, degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A. et al., “Enhanced evolutionary PCR using oligonucleotides with inosine at the 3′-terminus,” Nucleic Acid Res., Vol. 19, No. 18, pp. 5081, 1991; Ohtsuka, E. et al., “An alternative approach to deoxyoligonucleotides as hybridization probes by insertion of deoxyinosine at ambiguous codon positions,” J. Biol. Chem., Vol. 260, No. 5, pp. 2605-2608, 1985; Rossolini, G. M. et al., “Use of deoxyinosine-containing primers vs degenerate primers for polymerase chain reaction based on ambiguous sequence information,” Mol. Cell. Probes, Vol. 8, No. 2, pp. 91-98, 1994).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine. Amino acid analogs refer to compounds that have the same basic chemical structure as a naturally occurring amino acid, for example, an alpha-carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, for example, Creighton, T. E., “Proteins—Structures and Molecular Properties,” W. H. Freeman & Co. Ltd., 1984).

The term “percentage of sequence identity” can be determined by comparing two optimally aligned sequences over a comparison window, wherein the portion of the polynucleotide sequence in the comparison window may comprise additions or deletions (i.e., gaps) as compared to the reference sequence (for example, a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Some embodiments provided herein provide polypeptides or polynucleotides that are substantially identical to the polypeptides or polynucleotides, respectively, exemplified herein. Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, for example, amino acid sequences of 20 or fewer amino acids, in some embodiments, substantial identity exists when one or two amino acid residues are conservatively substituted, according to the conservative substitutions defined herein.

In some embodiments, the percent identity is over a portion of an antibody noted herein (the framework region, C_{H}3, and/or Fc region). In such situations, the percent identity of the portion of the polypeptide can be identified separately from the rest of the protein or nucleic acid sequence. Thus, two portions (e.g., two FR, C_{H}3, and/or Fc region) can have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), while allowing for the remainder of the protein to either stay 100% identical to the comparison protein, our while also allowing the remainder of the protein to also have variation by a specified percent identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, for example, by the local homology algorithm of Smith and Waterman (1970) Adv. Appl. Math. 2:482c, by the homology alignment algorithm of Needleman, S. B. et al., “A general method applicable to the search for similarities in the amino acid sequence of two proteins,” J. Mol. Biol., Vol. 48, No. 3, pp. 443-453, 1970, by the search for similarity method of Pearson, W. R. et al., “Improved tools for biological sequence comparison,” Proc. Natl. Acad. Sci. U.S.A., Vol. 85, No. 8, pp. 2444-2448, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, for example, Ausubel, F. M. et al., Current Protocols in Molecular Biology, Supplement, 1995).

Two examples of algorithms that are suitable for determining percent sequence identity and sequence similarity are the BLAST and BLAST 2.0 algorithms, which are described in Altschul, S. F. et al., “Gapped BLAST and PSI-BLAST: a new generation of protein database search programs,” Nucleic Acids Res., Vol. 25, No. 17, pp. 3389-3402, 1977, and Altschul, S. F. et al., “Basic local alignment search tool,” J. Mol. Biol., Vol. 215, No. 3, pp. 403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, S. F. et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see, Henikoff, S. et al., “Amino acid substitution matrices from protein blocks,” Proc. Natl. Acad. Sci. U.S.A., Vol. 89, No. 22, pp. 10915-10919, 1992) alignments (B) of 50, expectation (E) of 10, M=5, N=−4, and a comparison of both strands.

The BLAST algorithm also performs a statistical analysis of the similarity between two sequences (see, for example, Karlin, S. et al., “Applications and statistics for multiple high-scoring segments in molecular sequences,” Proc. Natl. Acad. Sci. U.S.A., Vol. 90, No. 12, pp. 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, in some embodiments, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “cytotoxic agent” as used herein refers to a substance that inhibits or prevents a cellular function and/or causes cell death or destruction. The term is intended to include non-radioactive payloads (e.g., ADC), radioactive isotopes (e.g., ¹⁷⁷Lu, ²²⁵Ac, ⁶⁷Cu, ²²⁷Th, ²¹¹At, ¹³¹I, ¹²⁵I, ⁹⁰Y, ¹⁸⁶Re, ¹⁸⁸Re, ¹⁵³Sm, ²¹²Bi, ²¹³Bi, ³²P, ¹⁴⁹Tb, ¹⁶¹Tb, ²¹²Pb, and radioactive isotopes of Lu), chemotherapeutic agents (as defined elsewhere herein). Other cytotoxic agents are described below. A tumoricidal agent causes destruction of tumor cells.

A “toxin” is any substance capable of having a detrimental effect on the growth or proliferation of a cell. Non-radioactive payloads include those commonly used for antibody drug conjugates (ADC) and fragment drug conjugates (FDC), such as toxins belonging to the families of auristatins, maytansines, maytansinoids, calicheamicins, duocarymycins, pyrrolobenzodiazepines dimers and amatoxins. A “therapeutic ion” refers to an electrically charged particle that that is useful in the treatment of a disorder related to a target molecule. Examples of therapeutic ions include ¹⁸F, ¹⁸F-FAC, ³²P, ³³P ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁷Th, and ²²⁵Ac. These are also options of therapeutic agents.

A “chemotherapeutic agent” is a chemical compound useful in the treatment of cancer. Examples of chemotherapeutic agents include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; metabolic inhibitor such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′″-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ docetaxel (Rhone-Poulene Rorer, Antony, France); chloranbucil; gemcitabine (GEMZAR™); 6-thioguanine; mercaptopurine; methotrexate; platinum analogs such as cisplatin and carboplatin; vinblastine (VELBAN™); platinum; etoposide (VP-16); ifosfamide; mitoxantrone; vincristine (ONCOVIN™); oxaliplatin; leucovovin; vinorelbine (NAVELBINE™); novantrone; edatrexate; daunomycin; aminopterin; ibandronate; topoisomerase inhibitor RFS 2000; difluoromethylornithine (DMFO); retinoids such as retinoic acid; capecitabine (XELODA™); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. These are also options of therapeutic agents.

The terms “subject,” “patient,” and “individual” interchangeably refer to an entity that is being examined and/or treated. This can include, for example, a mammal, for example, a human or a non-human primate mammal. The mammal can also be a laboratory mammal, for example, mouse, rat, rabbit, hamster. In some embodiments, the mammal can be an agricultural mammal (for example, equine, ovine, bovine, porcine, camelid) or domestic mammal (for example, canine, feline).

### Antibodies

As discussed herein, antibodies having enhanced biodistribution and/or pharmacokinetics obtained through disruption of one or more clusters of surface-exposed positively charged amino acids are provided. Provided herein is a variant antibody (e.g., minibody, cys-diabody) that includes at least one disrupted cluster of surface-exposed positively charged amino acids, where the variant antibody varies from an original antibody that includes an original cluster having at least two (e.g., 2, 3, 4, 5, 6 or more) surface-exposed, positively charged amino acids within about 30 angstroms of each other. The variant antibody can vary from the original antibody by having a substitution of at least one of the surface-exposed, positively charged amino acids of the original cluster with one or more negatively charged or non-charged amino acid. The substitution of the surface-exposed, positively charged amino acid(s) of the cluster can disrupt the original cluster (e.g., by having less surface-exposed positive charges at the corresponding location on the variant antibody compared to the original antibody). In some embodiments, the original or variant antibody is any antibody construct. In some embodiments, this disruption is especially useful for constructs equal in size to or smaller than a minibody. In some embodiments, the antibody is a cys-diabody. In some embodiments, the antibody is a scFv-Fc (e.g., a scFv fused to a Fc) or a Nanobody®-Fc (e.g., a camelid Nanobody® or single domain fragment fused to a Fc).

The distance between the surface-exposed positively charged amino acids in the cluster can be the distance between the center of mass of each amino acid residue in a suitable three-dimensional model of the antibody or a portion thereof. The positively charged amino acids of the cluster are surface exposed as determined by the Van der Waals radii, the Solvent Accessible Area, an isopotential surface representation of the electrostatic field as calculated by the Adaptive Poisson-Boltzmann Solver, or a combination thereof.

In general, the positively charged amino acids are amino acids that are positively charged under physiological conditions (e.g., physiological pH, or pH 5.6-7.5, pH 6.0-7.5, or pH 7.3-7.4). In some embodiments, the positively charged amino acids are selected from: arginine, lysine, and histidine. In some embodiments, the positively charged amino acids are selected from: arginine, and lysine. The amino group at the N-terminus of a polypeptide or protein can also provide a positive charge. The negatively charged or non-charged amino acids include amino acids that are not the positively charged amino acids under physiological conditions (e.g., physiological pH, or pH 5.6-7.5, pH 6.0-7.5, or pH 7.3-7.4). In some embodiments, the negatively charged amino acid is selected from: aspartate and glutamate. The carboxyl group at the C-terminus of a polypeptide or protein can also provide a negative charge. In some embodiments, the non-charged amino acid is selected from: alanine, glycine, asparagine, glutamine, isoleucine, leucine, methionine, valine, phenylalanine, tyrosine, tryptophan, serine, threonine, cysteine, and methionine. In some embodiments, the negatively charged or non-charged amino acid is glutamine.

The surface-exposed, positively charged amino acids within a cluster can form a continuous patch of positive charge on the surface (e.g., isopotential electrostatic surface) of the antibody (e.g., original antibody having the cluster). In some embodiments, the cluster has at least two (e.g., 2, 3, 4, 5, 6 or more) surface-exposed, positively charged amino acids within about 30, 25, 20, 15, 14, 13, 12, 11, 10, or 5 angstroms or less of each other, or within a distance of each other in a range defined by any two of the preceding values (e.g., 30-5 angstroms, 30-10 angstroms, 20-5 angstroms, 15-10 angstroms, 12-10 angstroms of each other). In some embodiments, the at least two surface-exposed, positively charged amino acids of the cluster are within about 15 angstroms of each other. In some embodiments, where a cluster includes 3 or more surface-exposed, positively charged amino acids, the distance between the two residues that are the furthest apart from each other is within about 30 angstroms, e.g., 25, 20, 15, 14, 13, 12, 11, 10, or about 5 angstroms or less, or within a distance in a range defined by any two of the preceding values (e.g., 30-5 angstroms, 30-10 angstroms, 20-5 angstroms, 15-10 angstroms, 12-10 angstroms). In some embodiments, there are no intervening surface-exposed, negatively charged amino acids between at least two (e.g., 2, 3, 4, 5, 6 or more) of the surface-exposed, positively charged amino acids of the cluster. In some embodiments, there are no surface-exposed, negatively charged amino acids between any two of the surface-exposed, positively charged amino acids of the cluster.

Also provided herein is a variant antibody that includes at least one disrupted cluster of positively charged amino acids, wherein the variant antibody varies from an original antibody that includes an original cluster having at least two (e.g., 2, 3, 4, 5, 6, or more) positively charged amino acids within 12 residues (e.g., linearly—along the peptide backbone) of each other. The variant antibody can vary from the original antibody by having a substitution of at least one of the positively charged amino acids of the original cluster with a negatively charged or non-charged amino acid, wherein the cluster is outside of any CDR of the original antibody. The substitution of the positively charged amino acid(s) can disrupt the original cluster (e.g., by having less surface-exposed positive charges at the corresponding location on the variant antibody compared to the original antibody). In some embodiments, the original cluster has at least two positively charged amino acids within 12, 11, 10, 9, 8, 7, 6, 5, 4, 3, 2, or 1 residues of each other. As used herein, two amino acids are within 1 residue of each other when they are immediately adjacent to each other in the polypeptide chain, and are within 2 residues of each other when there is one other amino acid residue between each other in the polypeptide chain, and so on. In some embodiments, the CDR regions and/or the framework regions are based on the Kabat definition. In some embodiments, the CDR regions and/or the framework regions are based on the AHo definition.

In some embodiments, the original cluster is disrupted where the number of surface-exposed, positively charged amino acids is reduced by at least one (e.g., by one, two, three or more) in the variant antibody compared to the original antibody. In some embodiments, the variant antibody varies from the original antibody by up to 3 (e.g., 1, 2, or 3) amino acid substitutions (e.g., with a negatively charged or non-charged amino acid) that disrupt the cluster. In some embodiments, the cluster is disrupted where the number of surface-exposed, positively charged amino acids is reduced by, by about, or by at least 30%, 40%, 50%, 60%, 70%, 80%, 90%, or by about 100%, or by a percentage in a range defined by any two of the preceding values (e.g., by 30-90%, 30-70%, 50-100%, 30-60%, etc.) in the variant antibody compared to the original antibody. In some embodiments, the original cluster is disrupted where the number of surface-exposed, positively charged amino acids is reduced by about 33% or more in the variant antibody compared to the original antibody. In some embodiments, the original cluster is disrupted where the number of surface-exposed, positively charged amino acids is reduced by about 66% or more in the variant antibody compared to the original antibody. In some embodiments, the variant antibody includes a disrupted cluster that has or has about 70%, 60%, 50%, 40%, 30%, 20%, 10%, or 0%, or a percentage in a range defined by any two of the preceding values (e.g., 0-70%, 30-70%, 0-40%, etc.) of the total positive charge attributed to the original cluster in the original antibody due to disruption of the cluster. For example and without limitation, the original antibody can have a cluster of 3 positively charged amino acids (e.g., lysines and/or arginines), and one of the positively charged amino acids can be substituted by a non-charged or negatively charged amino acid (e.g., glutamine) in the variant antibody, whereby the original cluster is disrupted. In some embodiments, the variant antibody includes a disrupted cluster that has about 66% or less total positive charge attributed to the cluster in the original antibody due to disruption of the cluster. In some embodiments, the variant antibody includes a disrupted cluster that has about 33% or less total positive charge attributed to the cluster in the original antibody due to disruption of the cluster. In some embodiments, the total positive charge attributed to the cluster is determined by use of algorithms suitable for solving the Poisson-Boltzmann equation such as the Adaptive Poisson-Boltzmann Solver (APBS).

The relevant features of amino acid residues (e.g., surface exposure, distance between amino acids) of the antibody can be determined using any suitable option. Suitable options include, without limitation, a resolved crystal structure, homology modeling, molecular dynamics simulation, Adaptive Poisson-Boltzmann Solver, Van der Waals radii, Solvent Accessible Area, or a combination thereof.

In some embodiments, at least two of the surface-exposed positively charged amino acids of the cluster in the original antibody are within a contiguous stretch of 15 or fewer (e.g., 15, 12, 10, 8, 7, 6, 5, 4, 3, or 2) residues in a polypeptide of the original antibody. In some embodiments, at least two of the surface-exposed positively charged amino acids of the cluster in the original antibody are at least 20, at least 25, at least 30, at least 40, at least 50, at least 60, least 75, at least 100, at least 150, at least 200, or more residues apart, or are apart by a number of residues in a range defined by any two of the preceding values (e.g., 20-200, 20-50, 40-75, 30-100, 50-150, 100-200, etc.), in a polypeptide of the original antibody.

The substitution of the surface-exposed positively charged amino acids of the original cluster can be in any suitable non-CDR region of the antibody (e.g., the original antibody). In some embodiments, the substitution of at least one of the surface-exposed positively charged amino acids of the original cluster is in a light chain variable region (V_{L}) framework region (FR) of the original antibody. In some embodiments, the substitution of at least one of the surface-exposed positively charged amino acids of the original cluster is in a V_{L} FR2 of the original antibody. The cluster of surface-exposed, positively charged amino acids that is disrupted in the antibody, e.g., variant antibody, can be in any suitable non-CDR region of the antibody (e.g., the original antibody). In some embodiments, the disrupted cluster is in a light chain variable region (V_{L}) of the antibody (e.g., in a framework region of V_{L}). In some embodiments, the disrupted cluster is in a V_{L} FR2 of the antibody, e.g., variant antibody.

In some embodiments, the original antibody includes a polypeptide, or original polypeptide, having an original sequence of X₁X₂X₃X₄X₅X₆X₇ (SEQ ID NO:2), where X₁ is a positively charged amino acid, where X₂, X₃, X₅, and X₆ are each independently a negatively charged or a non-charged amino acid, and where X₄ and X₇ are each independently any amino acid with the proviso that at least one is a positively charged amino acid, where the sequence is outside of any CDR of the antibody, and where the at least one of X₄ and X₇ that is a positively charged amino acid is surface exposed and is part of the original cluster. In some embodiments, the original sequence is in a V_{L} FR2 of the antibody. In some embodiments, the substitution comprises substitution of the at least one of X₄ and X₇ with a negatively charged or non-charged amino acid. In some embodiments, X₁ is part of the original cluster. In some embodiments, X₁ and the at least one of X₄ and X₇ that is a positively charged amino acid are part of the original cluster. In some embodiments, all of the positively charged amino acids of the sequence are part of the original cluster. In some embodiments, X₂, X₃, X₅, and X₆ are each independently any non-charged amino acid. The antibody, e.g., variant antibody, can include the original sequence of X₁X₂X₃X₄X₅X₆X₇ (SEQ ID NO:2) and further have the substitution of the at least one of the surface-exposed positively charged amino acids of the original cluster with a negatively charged or non-charged amino acid, that disrupts the original cluster. In some embodiments, the antibody, e.g., variant antibody, includes the original sequence of X₁X₂X₃X₄X₅X₆X₇ (SEQ ID NO:2), and substitution of at least one of X₄ and X₇ with a negatively charged or non-charged amino acid that disrupts the original cluster. In some embodiments, the antibody, e.g., variant antibody, includes the original sequence of X₁X₂X₃X₄X₅X₆X₇ (SEQ ID NO:2), and substitution of X₄ and X₇ with a negatively charged or non-charged amino acid that disrupts the original cluster. In some embodiments, the original antibody includes an original polypeptide that includes the sequence QQX₁X₂X₃X₄X₅X₆X₇ (SEQ ID NO:3) comprising the original sequence of X₁X₂X₃X₄X₅X₆X₇ (SEQ ID NO:2), as described herein.

In some embodiments, positive charges attributed to the original sequence (X₁X₂X₃X₄X₅X₆X₇ (SEQ ID NO:2)) is reduced by, by about, or by at least 30%, 35%, 40%, 45%, 50%, 55%, 60%, 65%, 70%, 75%, 80%, 90%, 95%, or about 100%, or a percentage in a range defined by any two of the preceding values (e.g., 30-95%, 30-40%, 30-50%, 30-70%, 60-70%, 60-100%, etc.), in the variant polypeptide. In some embodiments, the positive charges attributed to the original sequence (X₁X₂X₃X₄X₅X₆X₇ (SEQ ID NO:2)) is reduced by about 33% or more in the variant polypeptide. In some embodiments, the positive charges attributed to the original sequence (X₁X₂X₃X₄X₅X₆X₇ (SEQ ID NO:2)) is reduced by about 66% or more in the variant polypeptide. In some embodiments, X₇ is a positively charged amino acid. In some embodiments, X₄ is a positively charged amino acid. In some embodiments, the positively charged amino acid is a lysine, arginine, or histidine. In some embodiments, the positively charged amino acid is a lysine. In some embodiments, at least one of X₂ and X₆ is a proline. In some embodiments, both X₂ and X₆ are proline. In some embodiments, X₃ is a glycine or glutamate. In some embodiments, X₅ is an alanine, valine, serine, or proline.

In some embodiments, the original antibody includes at least one of the following original sequences: KX₂X₃KX₅ X₆K (SEQ ID NO:73), where X₂, X₃, X₅, and X₆ are each independently a negatively charged or a non-charged amino acid; KX₂X₃X₄X₅ X₆R (SEQ ID NO:74), where X₂, X₃, X₄, X₅, and X₆ are each independently a negatively charged or a non-charged amino acid; or KX₂X₃X₄X₅ X₆K (SEQ ID NO:75), where X₂, X₃, X₄, X₅, and X₆ are each independently a negatively charged or a non-charged amino acid. In some embodiments, the original sequence is one of: KPGKAPK (SEQ ID NO:5), KPGQAPR (SEQ ID NO:6), KPEKAPK (SEQ ID NO:7), KPGKVPK (SEQ ID NO:8), KPGQPPR (SEQ ID NO:9), KPGQSPR (SEQ ID NO:10), KPGLAPR (SEQ ID NO:11), or KPGQPPK (SEQ ID NO:12), corresponding to X₁X₂X₃X₄X₅X₆X₇ (SEQ ID NO:2). In some embodiments, the original sequence is KPGKAPK (SEQ ID NO:5) or KPGQAPR (SEQ ID NO:6), corresponding to X₁X₂X₃X₄X₅X₆X₇ (SEQ ID NO:2).

The original sequence can be in any suitable non-CDR region of the antibody (e.g., the original antibody). In some embodiments, the original sequence is in a light chain variable region (V_{L}) of the antibody (e.g., in a framework region of V_{L}). In some embodiments, the original sequence is in a V_{L} FR2 of the antibody, e.g., variant antibody.

In some embodiments, the negatively charged or non-charged amino acid with which the at least one of the surface-exposed, positively charged amino acids of the cluster is substituted is a glutamine (e.g., a K to Q, or R to Q substitution).

In some embodiments, the variant antibody includes at least one of the following comprising the substitution of at least one of the positively charged amino acids: KPGQAPK (SEQ ID NO:13), KPGQAPQ (SEQ ID NO:14), KPGQSPQ (SEQ ID NO:16), and QQKPGQSPQ (SEQ ID NO: 15), e.g., in the V_{L} FR2. In some embodiments, the variant antibody includes at least one of the following comprising the substitution of at least one of the positively charged amino acids: KPGQAPK (SEQ ID NO:13), KPGQAPQ (SEQ ID NO: 14), and QQKPGQSPQ (SEQ ID NO:15), e.g., in the V_{L} FR2. In some embodiments, the variant antibody comprises a V_{L} FR2 having an amino acid sequence of: WYQQKPGQAPQLLIY (SEQ ID NO: 17), WYQQKPGQSPQLLIY (SEQ ID NO: 18), or WYQQKPGQAPKLLIY (SEQ ID NO: 19).

Also provided is an antibody that includes a variant polypeptide that varies from an original polypeptide comprising an original sequence X₁X₂X₃X₄X₅X₆X₇ (SEQ ID NO: 1), wherein the original sequence is outside of any CDR of the antibody, wherein at least two of X₁, X₂, X₃, X₄, X₅, X₆, and X₇ are each independently a surface-exposed, positively charged amino acid and form a cluster of surface-exposed positively charged amino acids in the original polypeptide, wherein the variant polypeptide varies from the original polypeptide by having a modification of at least one of the at least two surface exposed, positively charged amino acids of the original sequence that reduces at least about 33% of positive charges attributed to the original sequence. In some embodiments, at least 33% of positive charges attributed to the original sequence is reduced in the variant polypeptide compared to the original polypeptide. In some embodiments, about 60% or more of positive charges attributed to the original sequence is reduced in the variant polypeptide. In some embodiments, the modification reduces about or at least about 33%, 40%, 50%, 60%, 70%, 80%, 90%, or about 100%, or a percentage in a range defined by any two of the preceding values (e.g., by 33-90%, 33-50%, 50-100%, 33-60%, etc.) of positive charges attributed to the sequence in the variant polypeptide compared to the original polypeptide. In some embodiments, the number of positively charged amino acid in the original sequence is reduced by one or more (e.g., at least 2, 3, 4, or 5 or more) in the variant polypeptide. In some embodiments, at least one (e.g., at least 2, 3, 4, 5, 6 or more) of the at least two positively charged amino acids of the original sequence is substituted with a negatively charged or non-charged amino acid in the variant polypeptide.

In some embodiments, at least X₄ and X₇ are each independently a positively charged amino acid. In some embodiments, the positively charged amino acid is a lysine, arginine, or histidine. In some embodiments, X₂, X₃, X₅, and X₆, are each independently any negatively charged or non-charged amino acid in the original polypeptide. In some embodiments, X₂, X₃, X₅, and X₆ are each independently any non-charged amino acid. In some embodiments, at least one of X₄ and X₇ is substituted with a negatively charged or non-charged amino acid to reduce the positive charges. In some embodiments, the negatively charged or non-charged amino acid with which the at least one of X₄ and X₇ is substituted is a glutamine (e.g., K to Q, or R to Q substitution). In some embodiments, the variant polypeptide includes an alanine or serine at a position corresponding to X₅ in the original sequence of the original polypeptide.

In some embodiments, the variant polypeptide includes a light chain variable region (V_{L}) of the antibody. The V_{L} can include the 3 CDRs and 4 FRs of the light chain. In some embodiments, the variant polypeptide can include the V_{L} and the heavy chain variable region (V_{H}) of the antibody, e.g., as a scFv fragment. In some embodiments, the variant polypeptide includes a scFv fragment. The V_{L} sequence (e.g., the V_{L} FR sequences) in the polypeptide can be based on a human germline sequence. For example and without limitation, the FR of the V_{L} is the corresponding FR of a human germline sequence, with no substitution, or up to 1, 2, 3, 4, or 5 or more substitutions thereto to disrupt a cluster of surface-exposed positive charges, independently in each FR. In some embodiments, there is up to 1, 2, 3, 4, or 5 substitutions in FR2 to disrupt a cluster of surface-exposed positive charges, as described herein, and no substitution in the other FR. In some embodiments, the V_{L} (e.g., the V_{L} FR sequences) is based on a human germline belonging to the IGKV1 family. In some embodiments, the FR of the V_{L} is the corresponding FR of a human germline sequence belonging to the IGKV1 family, with no substitution, or up to 1, 2, 3, 4, or 5 substitutions thereto to disrupt a cluster of surface-exposed positive charges, independently in each FR. In some embodiments, there is up to 1, 2, 3, 4, or 5 or more substitutions in FR2 to disrupt a cluster of surface-exposed positive charges, as described herein, and no substitution in the other FR. In some embodiments, the V_{L} (e.g., the V_{L} FR sequences) is based on a human germline sequence IGKV1-39*01. In some embodiments, the FR of the V_{L} is the corresponding FR of a human germline sequence IGKV1-39*01, with no substitution, or up to 1, 2, 3, 4, or 5 or more substitutions thereto to disrupt a cluster of surface-exposed positive charges, independently in each FR. In some embodiments, there is up to 1, 2, 3, 4, or 5 or more substitutions in FR2 to disrupt a cluster of surface-exposed positive charges, as described herein, and no substitution in the other FR. In some embodiments, the V_{L} (e.g., the V_{L} FR sequences) is based on a human germline belonging to the IGKV3 family. In some embodiments, the FR of the V_{L} is the corresponding FR of a human germline sequence belonging to the IGKV3 family, with no substitution, or up to 1, 2, 3, 4, or 5 or more substitutions thereto to disrupt a cluster of surface-exposed positive charges, independently in each FR. In some embodiments, there is up to 1, 2, 3, 4, or 5 or more substitutions in FR2 to disrupt a cluster of surface-exposed positive charges, as described herein, and no substitution in the other FR. In some embodiments, the V_{L} (e.g., the V_{L} FR sequences) is based on a human germline sequence IGKV3-15*01. In some embodiments, the FR of the V_{L} is the corresponding FR of a human germline sequence IGKV3-15*01, with no substitution, or up to 1, 2, 3, 4, or 5 or more substitutions thereto to disrupt a cluster of surface-exposed positive charges, independently in each FR. In some embodiments, there is up to 1, 2, 3, 4, or 5 or more substitutions in FR2 to disrupt a cluster of surface-exposed positive charges, as described herein, and no substitution in the other FR. In some embodiments, the V_{L} (e.g., the V_{L} FR sequences) is based on a human germline sequence from subgroup 1 from both, the proximal and distal clusters: IGKV1, IGKV1D. In some embodiments, the V_{L} (e.g., the V_{L} FR sequences) is based on a human germline from subgroups 2-7 from both, the proximal and distal clusters: IGKV2, IGKV3, IGKV4, IGKV5, IGKV6, IGKV7, IGKV2D, IGKV3D, IGKV6D. As used herein, a variable region of an antibody of the present disclosure that is “based on” a reference sequence (e.g., a human germline sequence) denotes that the framework regions (e.g., FR1, FR2, FR3, and FR4) of the antibody have sequences that are derived from the corresponding framework regions of the reference sequence (e.g., a human germline sequence), with no substitution, or up to 1, 2, 3, 4, or 5 or more substitutions thereto to disrupt a cluster of surface-exposed positive charges, independently in each FR. In some embodiments, there is up to 1, 2, 3, 4, or 5 or more substitutions in FR2 to disrupt a cluster of surface-exposed positive charges, as described herein, and no substitution in the other FR. In some embodiments, a variable region of an antibody that is based on a reference sequence (e.g., a human germline sequence) has framework regions that are, are about, or are at least 80%, 85%, 90%, 95%, or about 100%, or a percentage in a range defined by any two of the preceding values (e.g., 80-100%, 85-95%, 90-100%, etc.) identical to the corresponding framework regions of the reference sequence (e.g., a human germline sequence). In some embodiments, a variable region of an antibody that is based on a reference sequence (e.g., a human germline sequence) has framework regions having the sequence of the corresponding framework regions of the reference sequence (e.g., a human germline sequence) with up to 8, 7, 6, 5, 4, 3, 2, 1, or no substitutions thereto.

In some embodiments, the cluster is within a V_{L} framework region (FR) of the antibody. In some embodiments, the cluster is within a V_{L} FR2 of the antibody.

In some embodiments, the original polypeptide is based on a first human germline sequence of a V_{L} having an original FR2 comprising the original sequence, and wherein the variant polypeptide varies from the original polypeptide by having a substitution of at least the original sequence with a corresponding, second sequence from a second FR2 from a second human germline sequence of a V_{L}, wherein the second FR2 comprises the second sequence corresponding to the original sequence and having at least about 33% less positive charge attributed thereto compared to the original sequence. For example, a V_{L} of an antibody of the present disclosure may be based on an original V_{L} having an FR2 containing the cluster of positively charged amino acids. In a non-limiting example, the original V_{L} can be based on germline IGKV1-39*01, having the original sequence KPGKAPK (SEQ ID NO:5) in FR2. To disrupt the cluster of positively charged amino acids in the original antibody, key residues in FR2 are substituted with the corresponding residues in FR2 of germline IGKV2-28*01, having the original sequence KPGQSPQ (SEQ ID NO: 16). In some embodiments, this involves making at least three substitutions to the original FR2 sequence. In some embodiments, a subsequence of the FR2 of at least 3, 4, 5, 6, 7 or more residues is substituted to disrupt the cluster of positively charged amino acids. In some embodiments, the entire FR2 is substituted to disrupt the cluster of positively charged amino acids.

The first human germline sequence can be any suitable human germline sequence that has a cluster of at least two positively charged amino acids in the original sequence X₁X₂X₃X₄X₅X₆X₇ (SEQ ID NO:1), e.g., in a V_{L} FR2, as discussed above. The second human germline sequence can be any suitable human germline sequence that has a disrupted cluster in the V_{L} FR2. Suitable second human germline sequences include, without limitation, IGKV2-28*01. In some embodiments, the second human germline sequence includes, without limitation, one of IGKV2-29*02, IGKV2D-29*02, IGLV3-1*01, IGKV3-19*01, IGKV2-28*01, IGLV6-57*01, IGKV1-40*01, IGLV3-21*02, and IGLV3-21*03.

In some embodiments, the antibody (e.g., variant antibody) includes at least one of the following that includes the substitution of at least one of the positively charged amino acids: KPGQAPK (SEQ ID NO: 13), KPGQAPQ (SEQ ID NO:14). KPGQSPQ (SEQ ID NO:16), and QQKPGQSPQ (SEQ ID NO:15), e.g., in the V_{L} FR2. In some embodiments, the antibody (e.g., variant antibody) includes at least one of the following that includes the substitution of at least one of the positively charged amino acids: KPGQAPK (SEQ ID NO:13), KPGQAPQ (SEQ ID NO:14), and QQKPGQSPQ (SEQ ID NO:15), e.g., in the V_{L} FR2. In some embodiments, the antibody includes a V_{L} FR2 having an amino acid sequence of: WYQQKPGQAPQLLIY (SEQ ID NO: 17), WYQQKPGQSPQLLIY (SEQ ID NO: 18), or WYQQKPGQAPKLLIY (SEQ ID NO: 19).

In some embodiments, the cluster is in a hinge region, e.g., the upper hinge region, of the antibody. In some embodiments, the upper hinge region having the cluster (e.g., in the original antibody without substitution or modification as described herein) includes the original sequence EPKSSDKTHT (SEQ ID NO:38). In some embodiments, the antibody, e.g., the variant antibody, includes the upper hinge sequence of EPGSSDGTHT (SEQ ID NO:39).

In some embodiments, the antibody (e.g., the original antibody and/or the variant antibody) has a molecular weight (as a dimer of polypeptides, e.g., excluding any non-polypeptide features) of about 10, 20, 30, 40, 45, 50, 55, 60, 65, 70, 75, 80, 85, 90, 100 or about 110 kDa, or a molecular weight (as a dimer of polypeptides, e.g., excluding any non-polypeptide features) in a range defined by any two of the preceding values. In some embodiments, the antibody has a molecular weight (as a dimer of polypeptides, e.g., excluding any non-polypeptide features) of about 10-110 kDa, e.g., about 10-90 kDa, about 30-110 kDa, about 40-90 kDa, or about 30-80 kDa. In some embodiments, the antibody has a molecular weight (as a dimer of polypeptides, e.g., excluding any non-polypeptide features) of about 40-60 kDa, about 70-90 kDa, or about 45-85 kDa. In some embodiments, the antibody has a molecular weight (as a dimer of polypeptides, e.g., excluding any non-polypeptide features) of about 110 kDa or less, e.g., about 90 kDa or less, or about 80 kDa or less.

In some embodiments, the variant antibody is substantially identical to the original antibody other than the substitution of the at least one of the surface-exposed, positively charged amino acids of the cluster with a negatively charged or non-charged amino acid. In some embodiments, the variable regions of the variant antibody (e.g., V_{L} and V_{H}) are substantially identical to the original antibody other than the substitution of the at least one of the surface-exposed, positively charged amino acids of the cluster with a negatively charged or non-charged amino acid. In some embodiments, the pattern of isopotential electrostatic surfaces (e.g., patches of positive and negative charges) is substantially identical to the original antibody other than the reduction in surface-exposed positive charge attributed to the disrupted cluster of positively charged amino acids due to substitution of the at least one of the surface-exposed, positively charged amino acids of the cluster with a negatively charged or non-charged amino acid. In some embodiments, the variant antibody varies from the original antibody by up to 3 (e.g., 1, 2, or 3) amino acid substitutions. In some embodiments, the amino acid substitutions are outside of any CDR of the antibody.

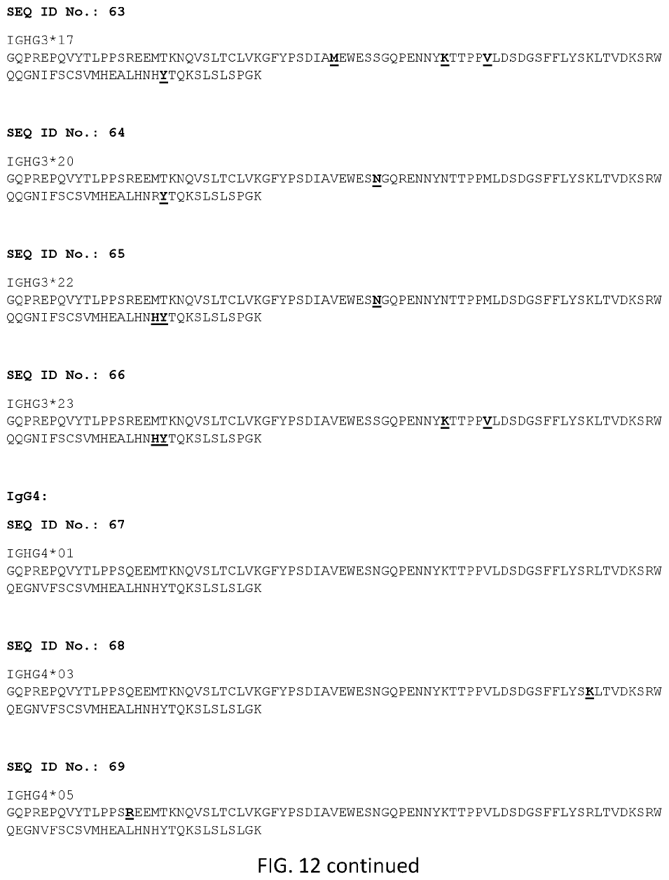

In some embodiments, the antibody (including, e.g., the original antibody, the variant antibody) includes an antigen-binding fragment, such as, without limitation, a scFv fragment. In some embodiments, the antibody is a minibody or a cys-diabody. In some embodiments, the antibody is a minibody. In some embodiments, the antibody is a minibody and includes a C_{H}3 having any one of the amino acid sequences shown in FIG. 12 (SEQ ID NOs:49-69). In some embodiments, the antibody is a cys-diabody.

Also provided herein is a minibody or cys-diabody comprising a variant polypeptide that varies from an original polypeptide that includes a light chain variable region (V_{L}) FR2 having an original sequence X₁X₂X₃X₄X₅X₆X₇ (SEQ ID NO: 1), wherein X₁, X₂, X₃, X₄, X₅, X₆, and X₇ are each independently any amino acid with the proviso that at least two are surface-exposed, positively charged amino acids that form a cluster of surface-exposed positively charged amino acids in the original polypeptide, where the variant polypeptide varies from the original polypeptide by having a modification of at least one of the at least two surface exposed, positively charged amino acids of the original sequence that reduces at least 33% of positive charges attributed to the original sequence. Suitable options for the original sequence X₁X₂X₃X₄X₅X₆X₇ (SEQ ID NO: 1) are as discussed herein.

In some embodiments, the negatively charged or non-charged amino acid with which the at least one of X₄ and X₇ is substituted is a glutamine (e.g., K to Q, or R to Q substitution). In some embodiments, the variant polypeptide includes an alanine or serine at a position corresponding to X₅ in the sequence of the original polypeptide.

Also provided is a minibody or cys-diabody that includes a light chain variable region (V_{L}) having a FR2 sequence of X₁X₂X₃QAX₆X₇, (SEQ ID NO:4), wherein X₁ is a surface-exposed, positively charged amino acid, wherein X₂, X₃, and X₆, are each independently any negatively charged or non-charged amino acid, and wherein X₇ is either glutamine or lysine. In some embodiments, X₂, X₃, and X₆ are each independently any non-charged amino acid. In some embodiments, the FR2 sequence includes KPGQAPK (SEQ ID NO: 13) or KPGQAPQ (SEQ ID NO: 14).

In some embodiments, X₁ is a lysine. In some embodiments, X₂ and/or X₆ is a proline. In some embodiments, X₂ and X₆ are each a proline. In some embodiments, X₃ is a glycine or glutamate.

In some embodiments, the minibody or cys-diabody includes at least one of KPGQAPK (SEQ ID NO: 13), KPGQAPQ (SEQ ID NO: 14), and QQKPGQSPQ (SEQ ID NO: 15) in the V_{L} FR2. In some embodiments, the minibody or cys-diabody includes at least one of KPGQAPK (SEQ ID NO:13), KPGQAPQ (SEQ ID NO:14), KPGQSPQ (SEQ ID NO:16), and QQKPGQSPQ (SEQ ID NO:15) in the V_{L} FR2. In some embodiments, the minibody or cys-diabody includes a V_{L} FR2 having an amino acid sequence of: WYQQKPGQAPQLLIY (SEQ ID NO: 17), WYQQKPGQSPQLLIY (SEQ ID NO: 18), or WYQQKPGQAPKLLIY (SEQ ID NO: 19).

Also provided herein is an antibody, minibody, or cys-diabody that includes a light chain variable region (V_{L},) including (by IMGT numbering): A49 and either: (i) Q51; or (ii) Q48 and K51. In some embodiments, the V_{L} further includes K45 (IMGT numbering). In some embodiments, the V_{L} further includes G47 (by IMGT numbering). Also provided herein is an antibody, minibody, or cys-diabody that includes a light chain variable region (V_{L}) including (by IMGT numbering): Q43, A49, and either: (i) Q51; or (ii) Q48 and K51. In some embodiments, the V_{L} further includes K45 (IMGT numbering). In some embodiments, the V_{L} further includes G47 (by IMGT numbering).

Also provided herein is a minibody having an upper hinge sequence of EPGSSDGTHT (SEQ ID NO:39). In some embodiments, the minibody includes an scFv linked to a C_{H}3 via a hinge region comprising an upper hinge sequence of EPGSSDGTHT (SEQ ID NO:39). In some embodiments, the minibody includes a hinge sequence of EPGSSDGTHTCPPCPPC (SEQ ID NO:71). In some embodiments, the minibody includes an scFv linked to a C_{H}3 via a hinge region comprising EPGSSDGTHTCPPCPPC (SEQ ID NO:71). In some embodiments, the minibody includes any one of the V_{L} FR2 sequences having a disrupted cluster of positively charged amino acids, as described herein.

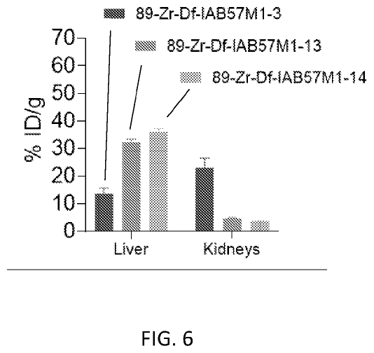

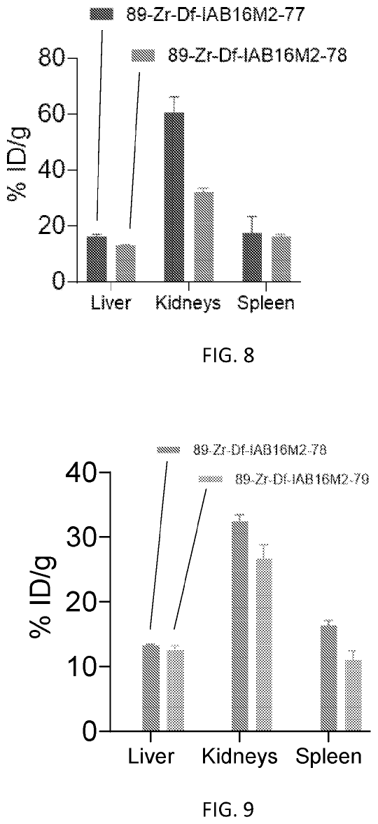

In any of the antibodies, minibodies, or cys-diabodies herein, in some embodiments, the antibody, minibody or cys-diabody (or the variant thereof) includes a light chain variable region (V_{L}) FR1, FR3, and FR4 of a human germline sequence from subgroup 1, from both the proximal and distal clusters: IGKV1, IGKV1D. In some embodiments, the V_{L} FR1, FR3, and FR4 are each from the corresponding V_{L} FR1, FR3, and FR4 of a human germline from subgroups 2-7 from both the proximal and distal clusters: IGKV2, IGKV3, IGKV4, IGKV5, IGKV6, IGKV7, IGKV2D, IGKV3D, IGKV6D. In some embodiments, the V_{L} FR1, FR3, and FR4 are each from the corresponding V_{L} FR1, FR3, and FR4 of a human germline IGKV1-39*01 or IGKV3-15*01. In some embodiments, the V_{L} FR1, FR3, and FR4 are each from the corresponding V_{L} FR1, FR3, and FR4 of any one of the sequences shown in FIGS. 5A-5C and 7A-7D. In some embodiments, the V_{L} FR1, FR3, and FR4 are each at least 80%, 85%, 90%, 95%, 97%, or about 100% identical, or identical by a percentage in a range defined by any two of the preceding values (e.g., 80-100%, 85-97%, 85-95%, 90-100%, etc.), to the corresponding V_{L} FR1, FR3, and FR4 of any one of the sequences shown in FIGS. 5A-5C and 7A-7D. In some embodiments, the antibodies, minibodies, or cys-diabodies (or the variant thereof) includes heavy chain variable region (V_{H}) framework sequences (FR1, FR2, FR3, and FR4) of any one of the sequences shown in FIGS. 5A-5C and 7A-7D. In some embodiments, the V_{H} FR1, FR2, FR3, and FR4 are each at least 80%, 85%, 90%, 95%, 97%, or about 100% identical, or identical by a percentage in a range defined by any two of the preceding values (e.g., 80-100%, 85-97%, 85-95%, 90-100%, etc.), to the corresponding V_{H}FR1, FR2, FR3, and FR4 of any one of the sequences shown in FIGS. 5A-5C and 7A-7D. In some embodiments, the V_{L} and V_{H} FR sequences of the antibodies, minibodies, or cys-diabodies (or the variant thereof) are paired or combined according to any one of the constructs shown in FIGS. 5A-5C and 7A-7D. In some embodiments, the framework regions are based on the Kabat definition. In some embodiments, the framework regions are based on the AHo definition.

In some embodiments, the antibody, minibody or cys-diabody (or the variant thereof) includes a light chain variable region (V_{L}) FR1 that includes residues 1-23 (excluding the signal peptide) of SEQ ID NO:42. In some embodiments, the antibody, minibody or cys-diabody (or the variant thereof) includes a V_{L} FR3 that includes residues 57-88 (excluding the signal peptide) of SEQ ID NO:42. In some embodiments, the antibody, minibody or cys-diabody (or the variant thereof) includes a V_{L} FR4 that includes residues 98-107 (excluding the signal peptide) of SEQ ID NO:42. In some embodiments, the antibody, minibody or cys-diabody (or the variant thereof) includes a light chain variable region (V_{L}) FR1 that includes residues 1-23 (excluding the signal peptide) of SEQ ID NO:42, a V_{L} FR3 that includes residues 57-88 (excluding the signal peptide) of SEQ ID NO:42, and a V_{L} FR4 that includes residues 98-107 (excluding the signal peptide) of SEQ ID NO:42. In some embodiments, the antibody, minibody or cys-diabody (or the variant thereof) includes a light chain variable region (V_{L}) FR1 that includes residues 1-30 of SEQ ID NO:45. In some embodiments, the antibody, minibody or cys-diabody (or the variant thereof) includes a V_{L} FR3 that includes residues 59-97 of SEQ ID NO:45. In some embodiments, the antibody, minibody or cys-diabody (or the variant thereof) includes a V_{L} FR4 that includes residues 109-119 of SEQ ID NO:45. In some embodiments, the antibody, minibody or cys-diabody (or the variant thereof) includes a light chain variable region (V_{L}) FR1 that includes residues 1-30 of SEQ ID NO:45, a V_{L} FR3 that includes residues 59-97 of SEQ ID NO:45, and a V_{L} FR4 that includes residues 109-119 of SEQ ID NO:45.

In any of the minibodies of the present disclosure, in some embodiments, the minibody includes a C_{H}3 having any one of the amino acid sequences shown in FIG. 12 (SEQ ID NOs:49-69). In some embodiments, the minibody includes a C_{H}3 having at least 80%, 85%, 90%, 95%, 97%, or about 100% identity, or percent identity in a range defined by any two of the preceding values (e.g., 80-100%, 85-97%, 85-95%, 90-100%, etc.), to any one of the amino acid sequences shown in FIG. 12 (SEQ ID NOs:49-69).

Also provided is an antibody that includes at least one disrupted cluster of positively charged amino acids, the cluster having at least three positively charged amino acids in a framework region (FR) of an original antibody, wherein the antibody varies from the original antibody having the cluster by having a substitution of at least one of the positively charged amino acids of the cluster with a negatively charged or non-charged amino acid, whereby the cluster is disrupted. In some embodiments, the number of positively charged amino acids is reduced by 1, 2, 3, or more residues to thereby provide the disrupted cluster in the antibody. In some embodiments, the cluster is in a V_{L} FR2 of the original antibody. In some embodiments, the antibody is a minibody or a cys-diabody.

In any of the antibodies, minibodies, or cys-diabodies (or variant thereof) of the present disclosure, in some embodiments, the antibody, minibody or cys-diabody binds specifically to any suitable antigen target. In some embodiments, the antibody, minibody, or cys-diabody specifically binds to DLL3, FAP, CD8, CD4, CD3, IFNγ, Integrin αVβ6, FOLRα, or PSMA. In some embodiments, the antibody, minibody, or cys-diabody (or variant thereof) includes a heavy chain having 3 heavy chain variable region CDR (HCDR) sequences (e.g., HCDR1, HCDR2, HCDR3) of the 3 corresponding HCDRs in any one of the sequences in FIGS. 5B, 5C, 7B-7D. In some embodiments, the antibody, minibody, or cys-diabody (or variant thereof) includes a light chain variable region having 3 light chain variable region CDR (LCDR) sequences (e.g., LCDR1, LCDR2, and LCDR3) of the 3 corresponding LCDRs in any one of the sequences in FIGS. 5B, 5C, 7B-7D, and a heavy chain having 3 heavy chain variable region CDR (HCDR) sequences (e.g., HCDR1, HCDR2, HCDR3) of the 3 corresponding HCDRs in any one of the sequences in FIGS. 5B, 5C, 7B-7D. In some embodiments, the antibody, minibody, or cys-diabody (or variant thereof) includes a V_{H} having an amino acid sequence of a V_{H} in any one of the sequences in FIGS. 5B, 5C, 7B-7D. In some embodiments, the antibody, minibody, or cys-diabody (or variant thereof) includes a V_{L} having an amino acid sequence of a V_{L} in any one of the sequences in FIGS. 5B, 5C, 7B-7D, and a V_{H} having an amino acid sequence of a V_{H} in any one of the sequences in FIGS. 5B, 5C, 7B-7D. In some embodiments, the antibody, minibody, or cys-diabody (or variant thereof) includes a V_{H} having an amino acid sequence at least about 80%, 85%, 90%, 95%, 97%, 98%, 99%, or about 100%, of a percentage in a range defined by any two of the preceding values (e.g., 80-100%, 80-90%, 85-99%, 90-97%, etc.) identical to a V_{H} in any one of the sequences in FIGS. 5B, 5C, 7B-7D. In some embodiments, the antibody, minibody, or cys-diabody (or variant thereof) includes a V_{L} having an amino acid sequence at least about 80%, 85%, 90%, 95%, 97%, 98%, 99%, or about 100%, of a percentage in a range defined by any two of the preceding values (e.g., 80-100%, 80-90%, 85-99%, 90-97%, etc.) identical to a V_{L} in any one of the sequences in FIGS. 5B, 5C, 7B-7D, and a V_{H} having an amino acid sequence at least about 80%, 85%, 90%, 95%, 97%, 98%, 99%, or about 100%, of a percentage in a range defined by any two of the preceding values (e.g., 80-100%, 80-90%, 85-99%, 90-97%, etc.) identical to a V_{H} in any one of the sequences in FIGS. 5B, 5C, 7B-7D. In some embodiments, the antibody, minibody, or cys-diabody (or variant thereof) includes an amino acid sequence at least about 80%, 85%, 90%, 95%, 97%, 98%, 99%, or about 100%, of a percentage in a range defined by any two of the preceding values (e.g., 80-100%, 80-90%, 85-99%, 90-97%, etc.) identical to any one of the sequences in FIGS. 5B, 5C, 7B-7D, with or without the signal peptide. In some embodiments, the antibody, minibody, or cys-diabody (or variant thereof) includes an amino acid sequence at least about 80%, 85%, 90%, 95%, 97%, 98%, 99%, or about 100%, of a percentage in a range defined by any two of the preceding values (e.g., 80-100%, 80-90%, 85-99%, 90-97%, etc.) identical to any one of the sequences in FIGS. 5B, 5C, 7B-7D without the signal peptide.

In any antibody, minibody, or cys-diabody of the present disclosure, in some embodiments, the antibody, minibody, or cys-diabody does not include SEQ ID NO:46. In some embodiments, the antibody, minibody, or cys-diabody does not include a V_{L} having an amino acid sequence of a V_{L} in SEQ ID NO:46. In some embodiments, the antibody, minibody, or cys-diabody does not bind to FAP (e.g., human FAP).

The antibody, minibody, or cys-diabody can have monovalent, bivalent, or multivalent antigen binding specificity. In some embodiments, the antibody, minibody, or cys-diabody is monovalent. In some embodiments, the antibody, minibody, or cys-diabody is bivalent. A bispecific antibody, minibody, or cys-diabody can have two different heavy/light chain pairs and/or it can recognize two different epitopes. In some embodiments, the antibody, minibody, or cys-diabody is multivalent.

In some embodiments, the antibody includes a Fc region (e.g., a scFv-Fc, a nanobody-Fc). In some embodiments, the antibody includes an amino acid sequence identical to the amino acid sequence of a native or naturally-occurring Fe region (e.g., a human IgG1 Fc region). In some embodiments, the Fc region includes an amino acid sequence of SEQ ID NO:76, as set forth below.

```
(SEQ ID NO: 76)
        230 240 250 260 270 280 290
        | | | | | | |
THTCPPCPAPELLGGPSVFLFPPKPKDTLMISRTPEVTCVVVDVSHEDPEVKFNWYVDGVEVHNAKTK
                  300 310 320 330 340 350 360
                  | | | | | | |
PREEQYNSTYRVVSVLTVLHQDWLNGKEYKCKVSNKALPAPIEKTISKAKGQPREPQVYTLPPSREEMTK
                  370 380 390 400 410 420 430
                  | | | | | | |
NQVSLTCLVKGFYPSDIAVEWESNGQPENNYKTTPPVLDSDGSFFLYSKLTVDKSRWQQGNVFSCSVMHE
                  440
                  |
ALHNHYTQKSLSLSPGK
```

In some embodiments, the Fc region includes an amino acid sequence at least 70, 80, 90, 95, 96, 97, 98, 99, or about 100% identical or identical by a percentage in a range defined by any two of the preceding values (e.g., 80-100%, 85-97%, 85-95%, 90-100%, etc.), to SEQ ID NO:76. In some embodiments, the Fc region includes one or more (e.g., 1, 2, 3, 4, 5, 6, 7, 8, 9, 10 or more) mutations (e.g., substitutions) that changes (e.g., increase or reduce) an effector function (e.g., FcγR binding) and/or binding to the Fc neonatal receptor (FcRn). In some embodiments, the Fc region includes an amino acid sequence of SEQ ID NO: 76, with one or more (e.g., 1, 2, 3, 4, 5, 6, 7, 8, 9, 10 or more) mutations (e.g., substitutions) that changes (e.g., increase or reduce) an effector function (e.g., FcγR binding) and/or binding to the Fe neonatal receptor (FcRn). In some embodiments, the Fc region includes one or more (e.g., 1, 2, 3, 4, 5, 6, 7, 8, 9, 10 or more) mutations (e.g., substitutions) that reduces an effector function (e.g., FcγR binding) and/or binding to the Fc neonatal receptor (FcRn). In some embodiments, the Fc region includes an amino acid sequence of SEQ ID NO:76, with one or more (e.g., 1, 2, 3, 4, 5, 6, 7, 8, 9, 10 or more) mutations (e.g., substitutions) that reduces an effector function (e.g., FcγR binding) and/or binding to the Fc neonatal receptor (FcRn). In some embodiments, the one or more mutations that reduces FcRn binding is a mutation (e.g., substitution) of any one or more (e.g., one, two or all three) of I253, H310, and H435 (EU numbering). In some embodiments, the one or more mutations that reduces binding to the FcRn is any one or more (e.g., one, two or all three) of I253A, H310A, and H435A (EU numbering). In some embodiments, the Fc region includes one or more (e.g., one, two or all three) of I253A, H310A, and H435A (EU numbering). In some embodiments, the one or more mutations that reduces binding to the Fc neonatal receptor is a mutation (e.g., substitution) of N297 (EU numbering). In some embodiments, the one or more mutations that reduces binding to the Fc neonatal receptor is N297Q, N297A or N297G (EU numbering). In some embodiments, the Fc region includes N297Q (EU numbering).

In some embodiments, the one or more mutations that reduces Fe effector function is a mutation (e.g., substitution) of any one or more of: L234, L235, G236, G237, P238, H268, K322, L328, P329, A330, P331. In some embodiments, the one or more mutations that reduces Fc effector function is any one or more of: L234A, L235A, G236R, G237A, P238S, H268A, K322A, L328R, P329G, A330S, P331S. Further non-limiting examples of known mutations that change Fc effector function may be found in Wilkinson and Hale (2022) Systematic analysis of the varied designs of 819 therapeutic antibodies and Fc fusion proteins assigned international nonproprietary names. MAbs. 2022 January-December; 14(1):2123299. doi: 10.1080/19420862.2022.2123299. In some embodiments, the Fc region does not include a C-terminal lysine.

In some embodiments, an antibody of the present disclosure is a scFv-Fc (e.g., a scFv fused to a Fc). In some embodiments, the scFv-Fc includes a variable light (V_{L}) domain linked to a variable heavy (V_{H}) domain, a hinge domain, and an Fc region. In some embodiments an antibody of the present disclosure is a Nanobody®-Fc (e.g., a camelid nanobody (or single domain fragment) fused to a Fc).

### Use as Imaging Agents

In any of the antibodies, minibodies, or cys-diabodies of the present disclosure, in some embodiments, the antibody, minibody or cys-diabody includes a detectable label. In some embodiments, the antibody, minibody or cys-diabody includes a radionuclide or an organic dye. In some embodiments, the radionuclide is one or more of: ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹¹At, ²¹²Bi, ²¹²Pb, ²¹³Bi, ²²³Ra, ²²⁵Ac, and ²²⁷Th. In some embodiments, exemplary Paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu. In some embodiments, the detectable label is a radionuclide such as Yttrium-90, Lutetium-177, Zirconium-89, Copper-64, Fluorine-18, Gallium-68 or Actinium-225. Additional embodiments of a radionuclide include Copper-67, Astatine-211, Lead-212, Bismuth-212, Bismuth-213, and Thorium-227. In some embodiments, treatment of a target cell with these radionuclides can result in cell damage and death to a target tissue.

In some embodiments, the detectable label is a bioluminescence or fluorescent compound Examples include, fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP), phycoerythrin, and the like), autoquenched fluorescent compounds that are activated by tumor-associated proteases, enzymes (e.g., luciferase, horseradish peroxidase, alkaline phosphatase, and the like), nanoparticles, biotin, digoxigenin or combination thereof.

In some embodiments, the organic dye is IndoCyanine Green (ICG) or one of the dyes that fluoresces in the near infrared region, such as IR800.

In some embodiments, any of the detectable labels, radionuclides, or organic dyes described herein can be conjugated to the antibody, minibody or cys-diabody. In some embodiments, the antibody, minibody or cys-diabody further includes a chelating ligand, or is conjugated to a chelating ligand. Examples of chelating ligands that may be used according to the embodiments herein include, but are not limited to, ethylenediaminetetraacetic acid (EDTA), diethylenetriaminepentaacetic acid (DTPA), dodecane tetraacetic acid (DOTA), 1,4,7,10-tetraazacyclododececane, 1-(glutaric acid)-4,7,10-triacetic acid (DOTAGA), 1,4,7-triazacyclononane-1,4,7-triacetic acid (NOTA), 1,4,7-triazacyclononane, 1-glutaric acid-5,7 acetic acid (NODAGA), 4-[2-(bis-carboxymethyl-amino)-ethyl]-7-carboxymethyl-[1,4,7]triazonan-1-yl-acetic acid (NETA), deferoxamine (Df, which may also be referred to as DFO), porphyrins, polyamines, crown ethers, bis-thiosemicarbazones, polyoximes, and like groups.

Also provided herein is a composition that includes any of the antibody, minibody or cys-diabody of the present disclosure; and a pharmaceutically acceptable carrier.

### Use as Therapeutic Agents

Any of the antibodies, minibodies, or cys-diabodies of the present disclosure, in some embodiments, can be comprised in a therapeutic composition or a pharmaceutical composition, e.g., for use in treating a subject in need of treating a disease or condition. In some embodiments, the therapeutic composition is for use in treating cancer. In some embodiments, a pharmaceutical composition comprises a pharmaceutically acceptable carrier such as a pharmaceutically acceptable buffer.

In some embodiments, an antibody, minibody, or cys-diabody, or a variant thereof as described herein, is conjugated to a therapeutic agent. A “therapeutic agent” as used herein is an atom, molecule, or compound that is useful in the treatment of a disorder related to a target molecule. Examples of therapeutic agents include, but are not limited to, drugs, chemotherapeutic agents, therapeutic antibodies and antibody fragments, toxins, radioisotopes, enzymes (for example, enzymes to cleave prodrugs to a cytotoxic agent at the site of the antigen binding construct binding), nucleases, hormones, immunomodulators, antisense oligonucleotides, chelators, boron compounds, photoactive agents and dyes, elastin-like polypeptides such as PLGA, and nanoparticles. Examples of disorders include those related to one or more target molecules.

In some embodiments, antibodies, minibodies, or cys-diabodies, or a variant thereof as described herein, are conjugated to a therapeutic agent. While minibodies, cys-diabodies, or other antigen binding fragments can have a shorter circulation half-life compared to a full-length antibody, in some embodiments, these formats can exhibit improved tumor penetration based on their smaller size and be therapeutically effective when appropriately armed with a cytotoxic drug or radioisotope. In some embodiments, an antibody, minibody, or cys-diabody, drug-conjugate approach can be employed. In some embodiments, a therapeutic approach includes radioimmunotherapy by attaching an appropriate radiolabel such as, Iodine-131, a beta-emitter or alpha-emitter, such as, Yttrium-90, Lutetium-177, Copper-67, Terbium-149, Terbium-161, Astatine-211, Lead-212, Bismuth-212, Actinium-225, Bismuth-213, and Thorium-227, which can deliver cell damage and death to a target tissue. In some embodiments, the radiolabel includes Terbium-149, Terbium-161, or Lead-212. In some embodiments, treatment with these fragments armed with a cytotoxic drug or radionuclide result in less nonspecific toxicity as they will be cleared from the body more rapidly.

In some embodiments, the label and/or therapeutic agent comprises ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁴⁹Tb, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁷Th and ²²⁵Ac, or any combination thereof.

In some embodiments, the antibody, minibody, and/or cys-diabody, or a variant thereof as described herein, are conjugated to a therapeutic agent such as a chemotherapeutic agent. Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as detectable markers in accordance with the embodiments of the disclosure include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte—colony stimulating factor, granulocyte macrophage-colony stimulating factor, hexamethylmelamine, hydrocortisone hydroxyurea, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

In some embodiments, the antibody, minibody, and/or cys-diabody, or a variant thereof as described herein, are conjugated to a cytotoxic agent (e.g., a toxin). A cytotoxic agent or toxin that may be used in accordance with the embodiments of the disclosure include, but are not limited to, Auristatin E, Auristatin F, Dolastatin 10, Dolastatin 15, combretastatin and their analogs, maytansinoid, calicheamicin, alpha-amanitin, pyrrolobenzodiazepine dimers, epothilones, duocarmycin and their analogs, tubulysin D, basillistatins, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Any of the antibodies, minibodies, or cys-diabodies described herein, or a variant thereof as described herein, may be further conjugated with one or more additional therapeutic agents, detectable markers, nanoparticles, carriers or a combination thereof. For example, an antigen binding construct may be radiolabeled with Iodine-131 and conjugated to a lipid carrier, such that the anti-target molecule-lipid conjugate forms a micelle. The micelle can incorporate one or more therapeutic or detectable markers.

The pharmaceutical, or therapeutic, compositions described herein can be administered by any suitable route of administration. A route of administration can refer to any administration pathway known in the art, including but not limited to aerosol, enteral, nasal, ophthalmic, oral, parenteral, rectal, transdermal (e.g., topical cream or ointment, patch), or vaginal. “Transdermal” administration can be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intracranial, intraventricular, intrauterine, intravenous, subarachnoid, subcapsular, sublingual, subcutaneous, transmucosal, or transtracheal. In some embodiments, the antigen binding construct can be delivered intraoperatively as a local administration during an intervention or resection.

### Nucleic Acids, Expression Vectors, Host Cells

Also provided herein are nucleic acids encoding the antibodies of the present disclosure. As used herein with reference to an antibody, minibody, or cys-diabody that include a dimer of monomer chains, “encoding an antibody, minibody, or cys-diabody” contemplates encoding at least one monomer of the antibody, minibody, or cys-diabody, where the nucleic acid encoding the monomer chain can generate, when expressed under suitable conditions, the dimeric antibody, minibody, or cys-diabody. In some embodiments, the nucleic acid encodes a variant polypeptide of the present disclosure.

In some embodiments, a nucleic acid is provided that encodes any of the antibody, minibody, or cys-diabody disclosed herein. In some embodiments, a nucleic acid is provided that encodes any of the variant polypeptides disclosed herein. In some embodiments, an expression vector is provided that comprises these nucleic acid sequences. In some embodiments, the expression vector includes pcDNA3.1™/myc-His (−) Version A vector for mammalian expression (Invitrogen, Inc.) or a variant thereof. The pcDNA3.1 expression vector features a CMV promoter for mammalian expression and both mammalian (Neomycin) and bacterial (Ampicillin) selection markers. In some embodiments, the expression vector includes a plasmid. In some embodiments, the vector includes a viral vector, for example a retroviral or adenoviral vector. In embodiments, the vector includes a cosmid, YAC, or BAC.

Also provided is a host cell, e.g., a genetically engineered host cell, that produce any of the antibodies, minibodies, or cys-diabodies described herein. The host cell can be a mammalian cell such as the HEK293, 293f, Epxi293 cell-lines, or the CHO-K1 cell line. In some embodiments, one or more of a wide variety of mammalian or non-mammalian expression systems are used to produce the antibodies, minibodies, or cys-diabodies disclosed herein including, but not limited to mammalian expression systems (for example, CHO-K1 cells), bacterial expression systems (for example, E. coli, B. subtilis), yeast expression systems (for example, Pichia, S. cerevisiae) or any other known expression system. Other systems can include insect cells and/or plant cells.

### Methods

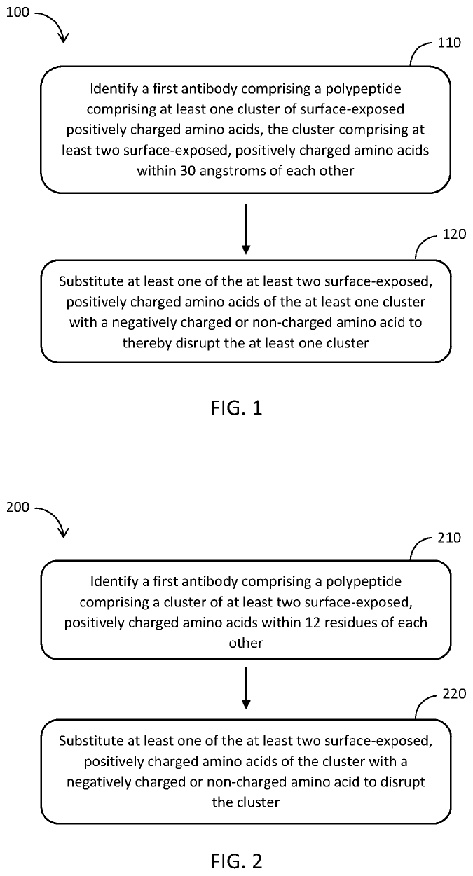

Also provided are methods for enhancing the biodistribution and/or pharmacokinetics of an antibody (e.g., minibody, cys-diabody) having a cluster of surface-exposed positively charged amino acids by disrupting the cluster. With reference to FIG. 1, a method of enhancing biodistribution and/or pharmacokinetics of an antibody, e.g., variant antibody, is provided. The method 100 can include, at block 110, identifying an original antibody having a polypeptide that includes at least one cluster of surface-exposed positively charged amino acids, the cluster comprising at least two (e.g., 2, 3, 4, 5, 6, or more) surface-exposed, positively charged amino acids within 30 angstroms of each other. The original antibody can be identified using any suitable option. In some embodiments, an Adaptive Poisson-Boltzmann Solver, Van der Waals radii, Solvent Accessible Area, or a combination thereof is used to identify an antibody having the at least one cluster of surface-exposed positively charged amino acids. In some embodiments, an antibody having the at least one cluster of surface-exposed positively charged amino acids is identified by analyzing the isopotential electrostatic surfaces of the antibody and calculating the linear distance between the center of mass of charged amino acid residues in a suitable model of the antibody (e.g., modeled using an Adaptive Poisson-Boltzmann Solver). The method can further include, at block 120, substituting at least one of the at least two surface-exposed, positively charged amino acids of the at least one cluster with a negatively charged or non-charged amino acid to thereby disrupt the at least one cluster. The variant antibody having the disrupted cluster of surface-exposed, positively charged amino acids can have enhanced biodistribution and/or pharmacokinetics compared to the original antibody. The original antibody can include any suitable cluster of surface-exposed positively charged amino acids as disclosed herein.

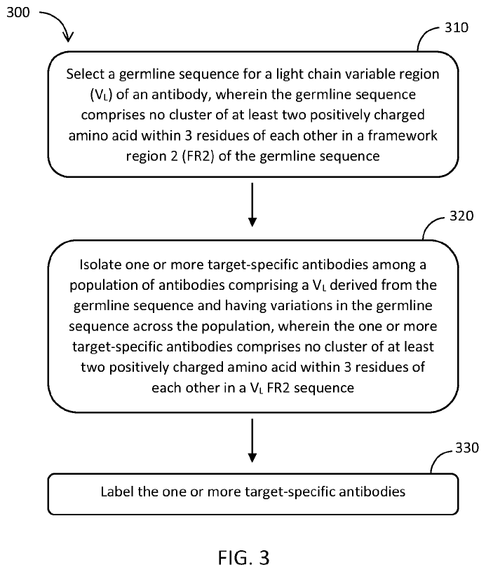

With reference to FIG. 2, a method of enhancing biodistribution and/or pharmacokinetics of an antibody is provided. The method 200 can include, at block 210, identifying an original antibody comprising a polypeptide comprising a cluster of at least two surface-exposed, positively charged amino acids within 12 residues of each other. The method can further include, at block 220, substituting at least one of the at least two surface-exposed, positively charged amino acids of the at least one cluster with a negatively charged or non-charged amino acid to thereby disrupt the at least one cluster. The variant antibody having the disrupted cluster of surface-exposed, positively charged amino acids can enhance biodistribution and/or pharmacokinetics compared to the original antibody. In some embodiments, the cluster includes the at least two positively charged amino acids within 6 residues of each other. In some embodiments, the cluster includes the at least two positively charged amino acids within 12, 11, 10, 9, 8, 7, 6, 5, 4, 3, 2 or 1 residue of each other.

In some embodiments, the polypeptide comprises the amino acid sequence of X₁X₂X₃X₄X₅X₆X₇ (SEQ ID NO: 1), wherein X₁, X₂, X₃, X₄, X₅, X₆, and X₇ are each independently any amino acid, wherein the at least two (e.g., 2, 3, 4, or more) surface-exposed, positively charged amino acids comprise at least two of X₁, X₂, X₃, X₄, X₅, X₆, and X₇. In some embodiments, the at least two surface-exposed, positively charged amino acids comprise at least X₁. In some embodiments, the at least two surface-exposed, positively charged amino acids comprise at least X₄ and X₇. In some embodiments, X₂, X₃, X₅, and X₆, are each independently any negatively charged or non-charged amino acid residue in the original polypeptide. In some embodiments, X₂, X₃, X₅, and X₆ are each independently any non-charged amino acid. In some embodiments, at least one of X₄ and X₇ are substituted with a negatively charged or non-charged amino acid. In some embodiments, each of X₄ and X₇ is independently substituted with a non-charged amino acid. In some embodiments, the at least two surface-exposed, positively charged amino acids comprise arginine, lysine and/or histidine.

In some embodiments, the negatively charged or non-charged amino acid with which the at least one of the at least two surface-exposed, positively charged amino acids is substituted includes a polar amino acid residue. In some embodiments, a polar amino acid is selected from: asparagine, glutamine, serine, threonine, and cysteine. In some embodiments, a polar amino acid is selected from: asparagine, glutamine, serine, and threonine. In some embodiments, the negatively charged or non-charged amino acid with which the at least one of the at least two surface-exposed, positively charged amino acids is substituted is glutamine. In some embodiments, the substituted positively charged amino acid of the cluster and the negatively charged or non-charged amino acid have similar side chain lengths. In some embodiments, the substituted positively charged amino acid of the cluster and the negatively charged or non-charged amino acid have side chain lengths with which the positively charged amino acid of the cluster is substituted have side chain lengths that are different by less than one or two carbons.

In some embodiments, the method further includes substituting up to 3 (e.g., 1, 2, or 3) amino acids of the polypeptide to disrupt the at least one cluster. In some embodiments, the amino acid substitutions are outside of any CDR of the antibody.

The cluster of positively charged amino acids are in the original antibody as described herein. In some embodiments, the cluster is within a V_{L} framework region (FR) of the original antibody. In some embodiments, the cluster is within a V_{L} FR2 of the original antibody. In some embodiments, the polypeptide having the cluster is based on a first human germline sequence of a V_{L} having an original FR2 that includes the cluster, and the method includes substituting the original FR2, or a portion thereof that includes the cluster, with a second FR2, or a corresponding portion thereof, from a second human germline sequence of a V_{L}, where the second FR2 includes at least one fewer surface-exposed, positively charged amino acids compared to the cluster. In some embodiments, a stretch of amino acids in the original FR2 (e.g., a stretch of 7 amino acids, such as X₁X₂X₃X₄X₅X₆X₇ (SEQ ID NO: 1)) is modified by one or more amino acid substitutions to match the corresponding stretch of amino acids in the second human germline sequence. In a non-limiting example, the original V_{L} can be based on germline IGKV1-39*01, having the sequence KPGKAPK (SEQ ID NO:5) in FR2. The method can include substituting key residues in the original FR2 with the corresponding residues in FR2 of germline IGKV2-28*01, having the sequence KPGQSPQ (SEQ ID NO:16), to disrupt the cluster of positively charged amino acids in the original antibody. This involves making at least three substitutions to the original FR2 sequence. In some embodiments, a subsequence of the FR2 of at least 3, 4, 5, 6, or more residues is substituted to disrupt the cluster of positively charged amino acids. In some embodiments, the entire FR2 is substituted to disrupt the cluster of positively charged amino acids.

In some embodiments, the method further includes conjugating a chelating ligand to the variant antibody. Any suitable chelating ligand, such as, but not limited to those disclosed herein, can be conjugated to the variant antibody. In some embodiments, the method includes labeling the antibody with a radionuclide, such as but not limited to those disclosed herein. In some embodiments, the method includes labeling the antibody with a detectable label, such as but not limited to those disclosed herein.

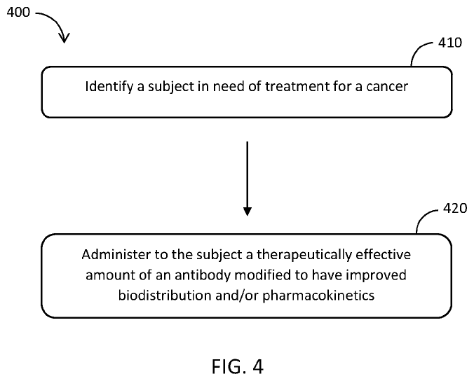

With reference to FIG. 3, a non-limiting method of making a labeled antibody is provided. The method 300 can include, at block 310, selecting a germline sequence (e.g., a human germline sequence) for a light chain variable region (V_{L}) of an antibody, wherein the germline sequence includes no cluster of at least two (e.g., 2 or 3) positively charged amino acids within 3 residues of each other in a framework region 2 (FR2) of the germline sequence. The germline sequence can be selected using any suitable option. In some embodiments, the germline sequence is selected by searching a database of human V_{L} germline sequences. In some embodiments, the germline sequence includes no cluster of at least two positively charged amino acids within 3, 4, 5, 6, 7, 8, 9, 10, or more residues of each other in a framework region 2 (FR2) of the germline sequence.

The method 300 can further include, at block 320, isolating one or more target-specific antibodies among a population of antibodies that includes a V_{L} derived from the germline sequence and having variations in the germline sequence across the population, wherein the one or more target-specific antibodies comprises no cluster of at least two positively charged amino acid within 3 residues of each other in a V_{L} FR2 sequence. The method can further include, at block 330, labeling the one or more target-specific antibodies.

The one or more target-specific antibodies can be isolated using any suitable option. In some embodiments, isolating the one or more target-specific antibodies includes screening a population of antibodies derived from the germline sequence. In some embodiments, the population of antibodies derived from the germline sequence are generated in a host organism genetically modified to express antibodies based only on the selected germline sequence (e.g., a mouse genetically modified to generate antibody clones based on a humanized or human germline sequence). In some embodiments, the population of antibodies derived from the germline sequence is part of a phage library of antibodies based only on the selected germline sequence. In some embodiments, the germline sequence is a human germline sequence. In some embodiments, the one or more target-specific antibodies are humanized.

In some embodiments, the antibody includes an antigen-binding fragment (e.g., an scFv fragment). The target-specific antibodies generated can be reformatted into a suitable antibody format (e.g., minibody or cys-diabody format). In some embodiments, the method includes generating one or more target-specific minibodies or cys-diabodies from the one or more target-specific antibodies.

In some embodiments, the label is a radionuclide, including but not limited to those disclosed herein. In some embodiments, labeling the one or more target-specific antibodies (including target specific minibodies or cys-diabodies) includes conjugating a chelating ligand to the one or more target-specific antibodies (or minibodies or cys-diabodies).

The antibodies (e.g., minibodies, cys-diabodies) of the present disclosure can be produced using any suitable option. In some embodiments, any method of enhancing an antibody of the present disclosure includes producing the antibody, e.g., variant antibody. In some embodiments, the antibodies (e.g., minibodies, cys-diabodies) are produced by expressing a nucleic acid encoding the antibody (e.g., encoding monomer chains of the antibody) in a suitable host cell expression system (e.g., mammalian cell line, bacteria, insect cell line, yeast, etc.).

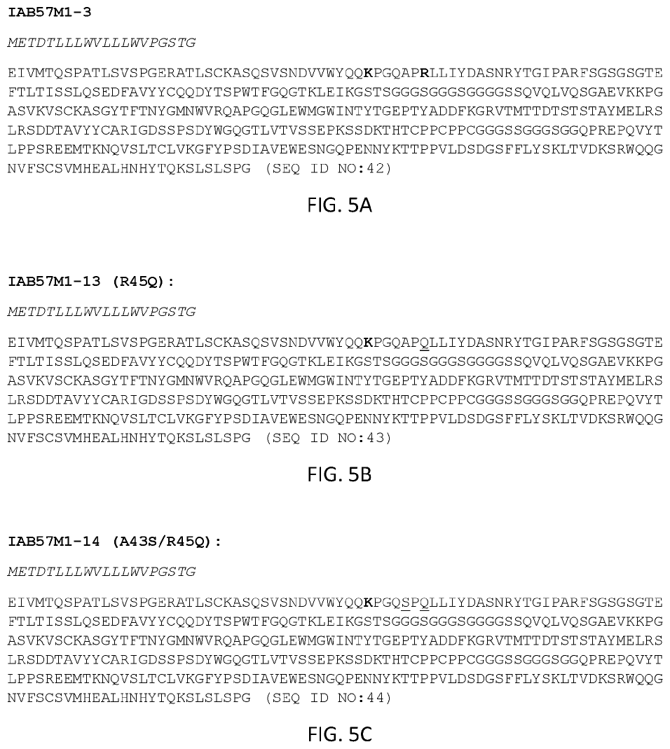

Also provided herein is a method of treating a subject using an antibody, minibody, or cys-diabody of the present disclosure. With reference to FIG. 4, a non-limiting method of treating a subject is provided. The method 400 can include, at block 410, identifying a subject in need of treatment, for example, for cancer, with the antibody, minibody, or cys-diabody, or variant thereof, of the present disclosure, and at block 420, administering a therapeutically effective amount of the antibody, minibody, or cys-diabody, to the subject. In some embodiments, the method includes administering to the subject a therapeutically effective amount of a composition, e.g., a pharmaceutical composition, containing the antibody, minibody, or cys-diabody of the present disclosure. In some embodiments, the method is a radiotherapy method, where the subject in need of radiotherapy (e.g., a subject having cancer) is identified, and the antibody, minibody, or cys-diabody administered to the subject is labeled with a radionuclide. In some embodiments, the subject has cancer. In some embodiments, the subject has cancer or other condition associated with the antigen to which the antibody, minibody, or cys-diabody specifically binds. In some embodiments, a greater dose of radiation can be administered using the antibody, minibody, or cys-diabody of the present disclosure, compared to the original antibody, minibody, or cys-diabody.

Also provided is a method of radioimmunotherapy. The method can include identifying a subject in need of radioimmunotherapy, and administering to the subject a therapeutically effective amount of the antibody, minibody, or cys-diabody, or variant thereof, of the present disclosure.

Also provided is a method treating a subject for a cancer. The method can include identifying a subject in need of treatment for a cancer; and administering to the subject a therapeutically effective amount of any of the antibody, minibody, or cys-diabody, or variant thereof, of the present disclosure, to thereby treat the cancer. In some embodiments, the cancer is non-small cell lung cancer (NSCLC), Small Cell Lung Cancer (SCLC), Thymic Carcinoma, Lymphoma, Myxoid/Round Cell Liposarcoma, Liposarcoma, Synovial Sarcoma, Recurrent Adult Soft Tissue Sarcoma, Gliosarcoma, Astrocytoma, Acute Myelogenous Leukemia (AML), Malignant Solitary Fibrous Tumor of the Pleura (MSFT), Penile Cancer, Diffuse Intrinsic Pontine Glioma (DIPG), Thyroid Carcinoma, Head and neck Squamous Carcinoma (SCCHN), Adenocarcinoma of the Lung, Vulvar Cancer (squamous cell carcinoma), Bladder Cancer, Cervical Squamous Cell Carcinoma, Germ Cell Tumors, Testicular Cancer, Pancreatic Ductal Adenocarcinoma, Pancreatic Adenocarcinoma, Non-Melanoma Skin Cancers, Retroperitoneal and Peritoneal Carcinoma, Melanoma, Unresectable or Metastatic Melanoma, Mucosal Melanoma of the Head and Neck, Uveal Melanoma, Non-Cutaneous Melanoma, Cutaneous T-Cell Lymphoma, Occult Primary tumors, Biliary Cancer, Gastrointestinal Stromal Tumors (GIST), Mesothelioma, Biphasic Mesothelioma, Malignant Pleural Mesothelioma, Kidney cancer, Myelodysplastic syndrome, Liver Hepatocellular Carcinoma, Esophageal and Esophagogastric Junction Carcinoma, Extrahepatic Bile Duct Adenocarcinoma, Small Intestinal Malignancies, Gastric Adenocarcinoma, Cholangiocarcinoma, Intrahepatic ad extrahepatic Cholangiocarcinomas, Ovarian Surface Epithelial Carcinomas, Non-epithelial and epithelial Ovarian cancers, Breast Carcinoma, Triple Negative Breast Cancer, Endometrial carcinoma, Uterine sarcoma, Bone Cancers, Colorectal Adenocarcinoma, Prostatic Adenocarcinoma, Hormone-Resistant Prostate Cancer (PC), Neuroendocrine Prostate Cancer (NEPC), Neuroendocrine tumors, Solid tumors, Follicular Lymphoma, Kaposi Sarcoma, Carcinoma of the Genitourinary Tract, Fallopian Tube Cancer, Malignant Glioma, Waldenstrom Macroglobulinemia, Richter Syndrome, Refractory Splenic Marginal Zone Lymphoma, Refractory Small Lymphocytic Lymphoma, Refractory Nodal Marginal Zone Lymphoma, Refractory Lymphoplasmacytic Lymphoma, Refractory Extranodal Marginal Zone Lymphoma of the Mucosa-Associated Lymphoid Tissue, Refractory Chronic Lymphocytic Leukemia, Multiple Myeloma, Hodgkin's Lymphoma, Non-Hodgkin's Lymphoma, Diffuse Large B-Cell Lymphoma, Nasopharyngeal Carcinoma, Gastroesophageal Junction Adenocarcinoma, renal cell carcinomas, colon carcinomas, Transitional cell carcinoma (TCC), urothelial carcinoma (UCC), glioblastoma multiforme (GBM), Gallbladder cancers, and Merkel Cell Carcinoma.

In some embodiments, the cancer is: Prostate cancer, Lung cancers, Melanoma, Breast malignancies, CNS and brain Malignancies, Skin malignancies, Occult Primary tumors, Kidney cancers, Gastrointestinal malignancies, Ovarian Neoplasms, Renal Cancers, Biliary Cancer, Bladder cancer, Esophageal Neoplasms, Cervical cancers, Solid tumors, Head and neck cancers, Urogenital Neoplasms, Germ Cell Tumors, Testicular Cancer, Pancreatic cancers, Glioma, Liver cancers, Malignant Neoplasms of the Bone, Colorectal cancers, Thyroid Cancer, Thoracic and respiratory tumors, Lymphomas, Male and female genitourinary Malignancies, Bile duct cancers, Hematological Malignancies, Multiple Myeloma, Gallbladder cancers, endocrine tumors, ocular cancers, and tumors of the hematopoietic and lymphoid tissues.

In some embodiments, the method is a method of imaging a subject, where the antibody, minibody, or cys-diabody is detectably labeled, and the method further includes imaging the subject to detect the labeled antibody, minibody, or cys-diabody in the subject. The subject can be imaged using any suitable option for the detecting the detectable label. In some embodiments, the imaging includes positron emission tomography (PET), computed tomography (CT), single-photon emission computed tomography (SPECT), magnetic resonance imaging (NMR), or detection of fluorescence emissions. In some embodiments, detection can be via near-infrared (NIR) imaging and/or Cerenkov luminescence imaging.

In some embodiments, the antibody, minibody, or cys-diabody specifically binds to an antigen target, such as, but not limited to those targets disclosed herein.

Additional embodiments of the present disclosure are provided in the following numbered arrangements.

1. A variant antibody comprising at least one disrupted cluster of surface-exposed positively charged amino acids, wherein the variant antibody varies from an original antibody comprising an original cluster comprising at least two surface-exposed, positively charged amino acids within about 30 angstroms of each other by having a substitution of at least one of the surface-exposed positively charged amino acids of the original cluster with a negatively charged or non-charged amino acid.

2. The variant antibody of arrangement 1, wherein the at least two surface-exposed, positively charged amino acids of the original cluster are within about 15 angstroms of each other.

3. The variant antibody of arrangement 1 or 2, wherein the variant antibody has at least about 10% less total positive charge attributed to the cluster compared to the original antibody due to disruption of the cluster.

4. The variant antibody of any one of the preceding arrangements, wherein the variant antibody varies from the original antibody by up to 3 amino acid substitutions that disrupt the cluster.

5. The variant antibody of any one of the preceding arrangements, wherein the substitution of at least one of the surface-exposed positively charged amino acids of the original cluster is in a light chain variable region (V_{L}) framework region (FR) of the original antibody.

6. The variant antibody of arrangement 5, wherein the substitution of at least one of the surface-exposed positively charged amino acids of the original cluster is in a V_{L} FR2 of the original antibody.

7. The variant antibody of any one of the preceding arrangements, wherein the original antibody comprises a polypeptide comprising an original sequence of X₁X₂X₃X₄X₅X₆X₇ (SEQ ID NO:2), wherein X₁ is a positively charged amino acid, wherein X₂, X₃, X₅, and X₆ are each independently a negatively charged or a non-charged amino acid, and wherein X₄ and X₇ are each independently any amino acid with the proviso that at least one is a positively charged amino acid, wherein the original sequence is outside of any CDR of the antibody, wherein the at least one of X₄ and X₇ that is a positively charged amino acid is surface exposed and is part of the original cluster.

8. The variant antibody of arrangement 7, wherein the positive charges attributed to the original sequence is reduced by about 33% or more in the variant antibody.

9. The variant antibody of arrangement 8, wherein the positive charges attributed to the original sequence is reduced by about 60% or more in the variant antibody.

10. The variant antibody of any one of arrangements 7-9, wherein the substitution comprises substitution of the at least one of X₄ and X₇ with a negatively charged or non-charged amino acid.

11. The variant antibody of any one of arrangements 7-10, wherein the polypeptide comprises QQX₁X₂X₃X₄X₅X₆X₇ (SEQ ID NO:3) comprising the original sequence of X₁X₂X₃X₄X₅X₆X₇ (SEQ ID NO:2).

12. The variant antibody of any one of arrangements 7-11, wherein X₇ is a positively charged amino acid.

13. The variant antibody of any one of arrangements 7-12, wherein X₄ is a positively charged amino acid.

14. The variant antibody of any one of arrangements 7-13, wherein the positively charged amino acid is a lysine, arginine, or histidine.

15. The variant antibody of arrangement 14, wherein the positively charged amino acid is a lysine or arginine.

16. The variant antibody of any one of arrangements 7-13, wherein X₂ and/or X₆ is a proline.

17. The variant antibody of any one of arrangements 7-14, wherein X₃ is a glycine or glutamate.

18. The variant antibody of any one of arrangements 7-15, wherein X₅ is an alanine, valine, serine, or proline.

19. The variant antibody of any one of arrangements 7-16, wherein the original sequence is one of KX₂X₃KX₅ X₆K (SEQ ID NO:73), wherein X₂, X₃, X₅, and X₆ are each independently a negatively charged or a non-charged amino acid; KX₂X₃X₄X₅ X₆R (SEQ ID NO:74), wherein X₂, X₃, X₄, X₅, and X₆ are each independently a negatively charged or a non-charged amino acid; or KX₂X₃X₄X₅ X₆K (SEQ ID NO:75), wherein X₂, X₃, X₄, X₅, and X₆ are each independently a negatively charged or a non-charged amino acid.

20. The variant antibody of any one of arrangements 7-17, wherein the original sequence is one of: KPGKAPK (SEQ ID NO:5), KPGQAPR (SEQ ID NO:6), KPEKAPK (SEQ ID NO:7), KPGKVPK (SEQ ID NO:8), KPGQPPR (SEQ ID NO:9), KPGQSPR (SEQ ID NO:10), KPGLAPR (SEQ ID NO: 11), or KPGQPPK (SEQ ID NO: 12).

21. The variant antibody of arrangement 18, wherein the original sequence is KPGKAPK (SEQ ID NO:5) or KPGQAPR (SEQ ID NO:6).

22. The variant antibody of any one of arrangements 7-19, wherein the negatively charged or non-charged amino acid with which the at least one of the surface-exposed, positively charged amino acids of the cluster is substituted is a glutamine.

23. The variant antibody of any one of arrangements 7-22, wherein the original sequence is in a light chain variable region (V_{L}) framework region (FR) of the original antibody.

24. The variant antibody of arrangement 23, wherein the original sequence is in a V_{L} FR2 of the original antibody.

25. The variant antibody of any one of the preceding arrangements, wherein the variant antibody comprises at least one of KPGQAPK (SEQ ID NO:13), KPGQAPQ (SEQ ID NO: 14), KPGQSPQ (SEQ ID NO: 16), and QQKPGQSPQ (SEQ ID NO: 15) in a V_{L} FR2.

26. The variant antibody of any one of the preceding arrangements, wherein the variant antibody comprises V_{L} FR2 having an amino acid sequence of: WYQQKPGQAPQLLIY (SEQ ID NO: 17), WYQQKPGQSPQLLIY (SEQ ID NO: 18), or WYQQKPGQAPKLLIY (SEQ ID NO: 19).

27. The method of any one of any one of the preceding arrangements, wherein the substitution of at least one of the surface-exposed positively charged amino acids of the original cluster is in a hinge region of the original antibody.

28. The method of arrangement 27, wherein the substitution of at least one of the surface-exposed positively charged amino acids of the original cluster is in an upper hinge region of the original antibody.

29. An antibody comprising a variant polypeptide that varies from an original polypeptide comprising an original sequence X₁X₂X₃X₄X₅X₆X₇ (SEQ ID NO:1), wherein the original sequence is outside of any CDR of the original polypeptide, wherein X₁, X₂, X₃, X₄, X₅, X₆, and X₇ are each independently any amino acid with the proviso that at least two are surface-exposed, positively charged amino acids that form a cluster of surface-exposed positively charged amino acids in the original polypeptide,
wherein the variant polypeptide varies from the original polypeptide by having a modification of at least one of the at least two surface exposed, positively charged amino acids of the original sequence that reduces at least 33% of positive charges attributed to the original sequence.

30. The antibody of arrangement 29, wherein the positive charges attributed to the original sequence is reduced by about 60% or more in the variant polypeptide.

31. The antibody of arrangement 29 or 30, wherein the number of positively charged amino acid in the original sequence is reduced by one or more in the variant polypeptide.

32. The antibody of any one of arrangements 29-31, wherein at least one of the at least two positively charged amino acids of the original sequence is substituted with a negatively charged or non-charged amino acid in the variant polypeptide.

33. The antibody of any one of arrangements 29-32, wherein at least X₄ and X₇ are each independently a positively charged amino acid.

34. The antibody of any one of arrangements 29-33, wherein the positively charged amino acid is a lysine, arginine, or histidine.

35. The antibody of any one of arrangements 29-34, wherein X₂, X₃, X₅, and X₆, are each independently a negatively charged or non-charged amino acid.

36. The antibody of any one of arrangements 29-35, wherein at least one of X₄ and X₇ is substituted with a negatively charged or non-charged amino acid to reduce the positive charges.

37. The antibody of arrangement 36, wherein the negatively charged or non-charged amino acid with which the at least one of X₄ and X₇ is substituted is a glutamine.

38. The antibody of any one of arrangements 29-37, wherein the variant polypeptide comprises an alanine or serine at a position corresponding to X₅ in the sequence of the original polypeptide.

39. The antibody of any one of arrangements 29-38, wherein the variant polypeptide comprises a light chain variable region (V_{L}) of the antibody.

40. The antibody of any one of arrangements 29-39, wherein the original sequence is within a V_{L} framework region (FR) of the original polypeptide.

41. The antibody of arrangement 40, wherein the original sequence is within a V_{L}FR2 of the original polypeptide.

42. The antibody of 40 or 41, wherein the original polypeptide is based on a first human germline sequence of a V_{L} having an original FR2 comprising the original sequence, and wherein the variant polypeptide varies from the original polypeptide by having a substitution of at least the original sequence with a corresponding, second sequence from a second FR2 from a second human germline sequence of a V_{L}, wherein the second FR2 comprises the second sequence corresponding to the original sequence and having at least 10% less positive charge attributed thereto compared to the original sequence.

43. A variant antibody comprising at least one disrupted cluster of positively charged amino acids, wherein the variant antibody varies from an original antibody comprising an original cluster comprising at least two positively charged amino acids within 12 residues of each other by having a substitution of at least one of the positively charged amino acids of the original cluster with a negatively charged or non-charged amino acid, wherein the original cluster is outside of any CDR of the original antibody.

44. The variant antibody of arrangement 43, wherein the original cluster is in a light chain variable region (V_{L}) FR of the original antibody.

45. The variant antibody of arrangement 44, wherein the original cluster is in a V_{L} FR2 of the original antibody

46. The variant antibody of arrangement 45, wherein the variant antibody comprises at least one of the following comprising the substitution of at least one of the positively charged amino acids: KPGQAPK (SEQ ID NO:13), KPGQAPQ (SEQ ID NO:14), and QQKPGQSPQ (SEQ ID NO: 15).

47. The variant antibody of arrangement 45, wherein the variant antibody comprises V_{L} FR2 comprising an amino acid sequence of: WYQQKPGQAPQLLIY (SEQ ID NO: 17), WYQQKPGQSPQLLIY (SEQ ID NO: 18), or WYQQKPGQAPKLLIY (SEQ ID NO:19).

48. The variant antibody of arrangement 43, wherein the original cluster is in a hinge region of the original antibody.

49. The variant antibody of arrangement 48, wherein the original cluster is in an upper hinge region of the original antibody.

50. The variant antibody of arrangement 48 or 49, wherein the original antibody comprises an upper hinge sequence of EPKSSDKTHT (SEQ ID NO:38).

51. The antibody of any one of arrangements 48-50, wherein the variant antibody comprises an upper hinge sequence of EPGSSDGTHT (SEQ ID NO:39).

52. The antibody of any one of the preceding arrangements, wherein the antibody comprises an antigen-binding fragment.

53. The antibody of any one of the preceding arrangements, wherein the molecular weight of the antibody is in a range of about 15 kDa to about 110 kDa.

54. The antibody of any one of the preceding arrangements, wherein the antibody is a minibody, a cys-diabody, a scFv, a scFv-Fc, or a nanobody-Fc.

55. The antibody of any one of the preceding arrangements, wherein the molecular weight of the antibody is in a range of about 10 kDa to about 20 kDa.

56. The antibody of arrangement 55, wherein the antibody is a nanobody.

57. The antibody of any one of arrangements 1-54, wherein the molecular weight of the antibody is greater than 90 kDa.

58. The antibody of arrangement 57, wherein the antibody is an scFv-Fc.

59. A minibody or cys-diabody comprising a variant polypeptide that varies from an original polypeptide comprising a light chain variable region (V_{L}) FR2 comprising an original sequence X₁X₂X₃X₄X₅X₆X₇ (SEQ ID NO:1), wherein X₁, X₂, X₃, X₄, X₅, X₆., and X₇ are each independently any amino acid with the proviso that at least two are surface-exposed, positively charged amino acids that form a cluster of surface-exposed positively charged amino acids in the original polypeptide,
wherein the variant polypeptide varies from the original polypeptide by having a modification of at least one of the at least two surface exposed, positively charged amino acids of the original sequence that reduces at least 33% of positive charges attributed to the original sequence.

60. The minibody or cys-diabody of arrangement 59, wherein at least X₄ and X₇ are each independently a positively charged amino acid in the original polypeptide.

61. The minibody or cys-diabody of arrangement 59 or 60, wherein X₂, X₃, X₅, and X₆, are each independently a negatively charged or non-charged amino acid in the original polypeptide.

62. The minibody or cys-diabody of any one of arrangements 59-61, wherein at least one of X₄ and X₇ is substituted with a negatively charged or non-charged amino acid to reduce the positive charges.

63. A minibody or cys-diabody comprising a light chain variable region (V_{L}) comprising a FR2 sequence comprising X₁X₂X₃QAX₆X₇ (SEQ ID NO:4), wherein X₁ is a surface-exposed, positively charged amino acid, wherein X₂, X₃, and X₆, are each independently any negatively charged or non-charged amino acid, and wherein X₇ is either glutamine or lysine.

64. The minibody or cys-diabody of any one of arrangements 59-63, wherein X₁ is a lysine.

65. The minibody or cys-diabody of any one of arrangements 59-64, wherein X₂ and/or X₆ is a proline.

66. The minibody or cys-diabody of any one of arrangements 59-65, wherein X₃ is a glycine or glutamate.

67. The minibody or cys-diabody of arrangement 63, wherein the FR2 sequence comprises KPGQAPK (SEQ ID NO:13) or KPGQAPQ (SEQ ID NO:14).

68. A minibody or cys-diabody comprising a light chain variable region (V_{L}) comprising (by IMGT numbering):
A49 and either:
   (i) Q51; or
   (ii) Q48 and K51.

69. The minibody or cys-diabody of arrangement 61, wherein the V_{L} further comprises K45 (IMGT numbering).

70. A minibody comprising an upper hinge sequence of EPGSSDGTHT (SEQ ID NO:39).

71. The antibody, minibody, or cys-diabody of any one of the preceding arrangements, wherein the antibody, minibody, or cys-diabody specifically binds to DLL3, FAP, CD8, CD4, CD3, IFNγ, integrin αVβ6, FOLRα, or PSMA.

72. The antibody, minibody, or cys-diabody of any one of the preceding arrangements, wherein the antibody, minibody, or cys-diabody comprises a light chain variable region comprising 3 LCDR sequences in any one of the sequences in FIGS. 5B, 5C, 7B-7D, and a heavy chain variable region (V_{H}) comprising 3 HCDR sequences in any one of the sequences in FIGS. 5B, 5C, 7B-7D.

73. The antibody, minibody, or cys-diabody of any one of the preceding arrangements, further comprising a detectable label.

74. The antibody, minibody, or cys-diabody of arrangement 73, wherein the antibody, minibody, or cys-diabody is labeled with a radionuclide or an organic dye.

75. The antibody, minibody, or cys-diabody of any one of arrangements 1-72, further comprising a therapeutic agent.

76. The antibody, minibody, or cys-diabody of arrangement 75, wherein the therapeutic agent is a cytotoxic agent.

77. The antibody, minibody, or cys-diabody of arrangement 75, wherein the therapeutic agent is a radionuclide.

78. The antibody, minibody, or cys-diabody of arrangement 75, wherein the radionuclide is selected from among ²¹²Pb, ¹⁴⁹Tb and ¹⁶¹Tb.

79. A composition comprising: an antibody, minibody or cys-diabody of any one of the preceding arrangements; and a pharmaceutically acceptable carrier.

80. A nucleic acid encoding the variant polypeptide of the antibody of any one of arrangements 29-42 or the minibody or cys-diabody of any one of arrangements 59-62.

81. A nucleic acid encoding the variant antibody of any one of arrangements 1-28 and 43-58, the antibody of any one of arrangements 29-42, or the minibody or cys-diabody of any one of arrangements 59-72.

82. A genetically engineered host cell comprising the nucleic acid of arrangement 80 or 81.

83. A method of enhancing biodistribution and/or pharmacokinetics of an antibody, comprising:
identifying an original antibody comprising a polypeptide comprising at least one cluster of surface-exposed positively charged amino acids, the cluster comprising at least two surface-exposed, positively charged amino acids within 30 angstroms of each other;
substituting at least one of the at least two surface-exposed, positively charged amino acids of the at least one cluster with a negatively charged or non-charged amino acid to thereby disrupt the at least one cluster,
whereby a variant antibody having enhanced biodistribution and/or pharmacokinetics compared to the original antibody is generated.

84. A method of enhancing biodistribution and/or pharmacokinetics of an antibody, comprising:
identifying an original antibody comprising a polypeptide comprising a cluster of at least two surface-exposed, positively charged amino acids within 12 residues of each other; and
substituting at least one of the at least two surface-exposed, positively charged amino acids of the cluster with a negatively charged or non-charged amino acid to disrupt the cluster, thereby generating a variant antibody having enhanced biodistribution and/or pharmacokinetics compared to the original antibody.

85. The method of arrangement 84, wherein the cluster comprises the at least two positively charged amino acids within 6 residues of each other.

86. The method of arrangement 84 or 85, wherein the polypeptide comprises an amino acid sequence of X₁X₂X₃X₄X₅X₆X₇ (SEQ ID NO: 1), wherein X₁, X₂, X₃, X₄, X₅, X₆, and X₇ are each independently any amino acid, wherein the at least two surface-exposed, positively charged amino acids comprise at least two of X₁, X₂, X₃, X₄, X₅, X₆, and X₇.

87. The method of arrangement 86, wherein the at least two surface-exposed, positively charged amino acids comprise at least X₁.

88. The method of arrangement 86 or 87, wherein the at least two surface-exposed, positively charged amino acids comprise at least X₄ and X₇.

89. The method of any one of arrangements 86-88, wherein X₂, X₃, X₅, and X₆, are each independently a negatively charged or non-charged amino acid residue in the original polypeptide.

90. The method of any one of arrangements 86-89, wherein at least one of X₄ and X₇ are substituted with a negatively charged or non-charged amino acid.

91. The method of any one of arrangements 83-90, wherein the at least two surface-exposed, positively charged amino acids comprise arginine, lysine, or histidine.

92. The method of any one of arrangements 83-91, wherein the negatively charged or non-charged amino acid with which the at least one of the at least two surface-exposed, positively charged amino acids is substituted comprises a polar amino acid.

93. The method of arrangement 92, wherein the negatively charged or non-charged amino acid with which the at least one of the at least two surface-exposed, positively charged amino acids is substituted is glutamine.

94. The method of any one of arrangements 83-93, wherein the positively charged amino acid and the negatively charged or non-charged amino acid have side chain lengths that are different by less than one or two carbons.

95. The method of any one of arrangements 83-94, comprising substituting up to 3 amino acids of the polypeptide to disrupt the at least one cluster.

96. The method of any one of arrangements 83-95, wherein the cluster is outside of any complementarity determining region (CDR) of the original antibody.

97. The method of any one of arrangements 83-96, wherein the cluster is in a light chain variable region (V_{L}) of the original antibody.

98. The method of any one of arrangements 83-97, wherein the at least two surface-exposed, positively charged amino acids are within a V_{L} framework region (FR) of the original antibody.

99. The method of any one of arrangements 83-98, wherein the at least two surface-exposed, positively charged amino acids are within a V_{L} FR2 of the original antibody.

100. The method of arrangement 98 or 99, wherein the polypeptide is based on a first human germline sequence of a V_{L} having an original FR2 comprising the at least two surface-exposed, positively charged amino acids are, and wherein the method comprises substituting the original FR2, or a portion thereof comprising the cluster, with a second FR2, or a corresponding portion thereof, from a second human germline sequence of a V_{L}, wherein the second FR2 comprises at least one fewer surface-exposed, positively charged amino acids compared to the cluster.

101. The method of arrangement 99 or 100, wherein the original sequence is one of KX₂X₃KX₅ X₆K (SEQ ID NO:73), wherein X₂, X₃, X₅, and X₆ are each independently a negatively charged or a non-charged amino acid; KX₂X₃X₄X₅ X₆R (SEQ ID NO:74), wherein X₂, X₃, X₄, X₅, and X₆ are each independently a negatively charged or a non-charged amino acid; or KX₂X₃X₄X₅ X₆K (SEQ ID NO:75), wherein X₂, X₃, X₄, X₅, and X₆ are each independently a negatively charged or a non-charged amino acid.

102. The method of arrangement 99 or 101, wherein the original polypeptide comprises one of: KPGKAPK (SEQ ID NO:5), KPGQAPR (SEQ ID NO:6), KPEKAPK (SEQ ID NO:7), KPGKVPK (SEQ ID NO:8), KPGQPPR (SEQ ID NO:9), KPGQSPR (SEQ ID NO:10), KPGLAPR (SEQ ID NO:11), or KPGQPPK (SEQ ID NO:12) in a V_{L} FR2.

103. The method of any one of arrangements 99-101, wherein the variant polypeptide comprises at least one of KPGQAPK (SEQ ID NO: 13), KPGQAPQ (SEQ ID NO: 14), KPGQSPQ (SEQ ID NO: 16), and QQKPGQSPQ (SEQ ID NO: 15) in a V_{L} FR2.

104. The method of any one of arrangements 99-101, wherein the variant polypeptide comprises at least one of: WYQQKPGQAPQLLIY (SEQ ID NO:17), WYQQKPGQSPQLLIY (SEQ ID NO:18), or WYQQKPGQAPKLLIY (SEQ ID NO:19) in a V_{L} FR2.

105. The method of any one of arrangements 83-104, further comprising conjugating a chelating ligand to the variant antibody.

106. The method of any one of arrangements 83-105, further comprising labeling the antibody with a radionuclide.

107. The method of any one of arrangements 83-105, further comprising labeling the antibody with a detectable label.

108. A method of making a labeled antibody, comprising:
selecting a germline sequence for a light chain variable region (V_{L}) of an antibody, wherein the germline sequence comprises no cluster of at least two positively charged amino acids within 3 residues of each other in a framework region 2 (FR2) of the germline sequence;
isolating one or more target-specific antibodies among a population of antibodies comprising a V_{L} derived from the germline sequence and having variations in the germline sequence across the population, wherein the one or more target-specific antibodies comprises no cluster of at least two positively charged amino acid within 3 residues of each other in a V_{L}FR2 sequence; and
labeling the one or more target-specific antibodies.

109. The method of arrangement 108, wherein isolating the one or more target-specific antibodies comprises screening a population of antibodies derived from the germline sequence.

110. The method of arrangement 109, wherein the population of antibodies derived from the germline sequence are generated in a host organism genetically modified to express antibodies based only on the selected germline sequence.

111. The method of arrangement 109, wherein the population of antibodies derived from the germline sequence is comprised in a phage library of antibodies based only on the selected germline sequence.

112. The method of any one of arrangements 109-111, wherein the germline sequence is a human germline sequence.

113. The method of any one of arrangements 108-112, wherein the one or more target-specific antibodies are humanized.

114. The method of any one of arrangements 108-113, further comprising generating one or more target-specific minibodies or cys-diabodies from the one or more target-specific antibodies.

115. The method of any one of arrangements 108-114, comprising labeling the one or more target-specific antibodies with a radionuclide.

116. The method of any one of arrangements 108-115, wherein labeling the one or more target-specific antibodies comprises conjugating a chelating ligand to the one or more target-specific antibodies.

117. The method of any one of arrangements 83-116, wherein the antibody comprises an antigen-binding fragment.

118. The method of any one of arrangements 83-117, wherein the antibody is a minibody or a cys-diabody.

119. The method of any one of arrangements 83-118, wherein the antibody specifically binds to DLL3, FAP, CD8, CD4, CD3, IFNγ, integrin αVβ6, FOLRα, or PSMA.

120. The method of any one of arrangements 83-118, wherein the antibody or minibody comprises a light chain variable region (V_{L}) comprising 3 LCDR sequences of the 3 LCDRs of any one of the sequences in FIGS. 5A-5C and 7A-7D, and a heavy chain variable region (V_{H}) comprising the 3 HCDR sequences of 3 HCDRs of any one of the sequences in FIGS. 5A-5C and 7A-7D.

121. An antibody made by the method of any one of arrangements 83-120.

122. A method of treating a subject, comprising:
identifying a subject in need of treatment with the antibody, minibody, or cys-diabody of any one of arrangements 1-78; and
administering a therapeutically effective amount of the antibody or minibody, or the composition of arrangement 79 to the subject.

123. A method of treating a subject for a cancer, comprising:
identifying a subject in need of treatment for a cancer; and
administering to the subject a therapeutically effective amount of the antibody, minibody, or cys-diabody of any one of arrangements 1-78, or the composition of arrangement 79, to thereby treat the cancer.

124. A method of radiotherapy, comprising:
identifying a subject in need of radiotherapy; and
administering to the subject a therapeutically effective amount of the antibody, minibody, or cys-diabody of any one of arrangements 1-78, or the composition of arrangement 79, wherein the antibody, minibody, or cys-diabody comprises a radionuclide.

125. A method of imaging a subject, comprising:
administering to a subject a composition comprising an effective amount of the antibody, minibody, or cys-diabody of any one of arrangements 1-78 or the composition of arrangement 79, wherein the antibody, minibody, or cys-diabody is detectably labeled; and
imaging the subject to detect the labeled antibody, minibody, or cys-diabody in the subject.

126. Use of the antibody, minibody, or cys-diabody of any one of arrangements 1-78 or the composition of arrangement 79, for treatment of cancer in a subject in need thereof.

127. Use of the antibody, minibody, or cys-diabody of any one of arrangements 1-78 for preparation of a medicament for treatment of cancer in a subject in need thereof.

### EXAMPLES

### Example 1

This non-limiting example shows enhancing biodistribution of a minibody by mutating positively charged amino acids in a cluster of positively charged amino acids in the FR2 of the light chain variable region.

A minibody that binds to DLL3 was constructed by fusing a humanized heavy chain variable region (V_{H}) of an DLL3 antibody to the C-terminus of a humanized light chain variable region (V_{L}) of the DLL3 antibody via a linker to form an scFv fragment, and fusing the scFv fragment to a C_{H}3 domain via a hinge region. The amino acid sequence of the DLL3 minibody (IAB57M1-3) monomer is shown in FIG. 5A. In FIGS. 5A-5C, the signal peptide is shown in italics. The DLL3 minibody can be used to image in vivo DLL3-expressing tumor cells in a patient by conjugating a suitable chelator (e.g., Df) and labeling with a radionuclide (e.g., ⁸⁹Zr).

The humanized V_{L} used the germline sequence of IGVK3-15*01. The V_{L} has a framework region 2 (FR2) sequence (WYQQKPGQAPRLLI (SEQ ID NO:70) (according to North)) that includes a cluster of two basic amino acids (lysine and arginine) at positions 45 and 51 (IMGT numbering). Molecular modeling of the surface charges using an Adaptive Poisson-Boltzmann Solver identified a localized patch of surface-exposed positive charges formed by the two basic amino acids. The localized positively charged patch may contribute to accumulation of minibodies in the kidney.

To test this, R51 was substituted with a glutamine (Q), as shown in FIG. 5B. Thus, KPGQAPR (SEQ ID NO:6) in V_{L} FR2 of the original minibody was altered to KPGQAPQ (SEQ ID NO: 14) in the first variant. A second variant was made by further substituting the alanine (A) at position 49 with serine (S), as shown in FIG. 5C. Thus, KPGQAPR (SEQ ID NO:6) in V_{L} FR2 of the original minibody was further altered to KPGQSPQ (SEQ ID NO:16) in the second variant.

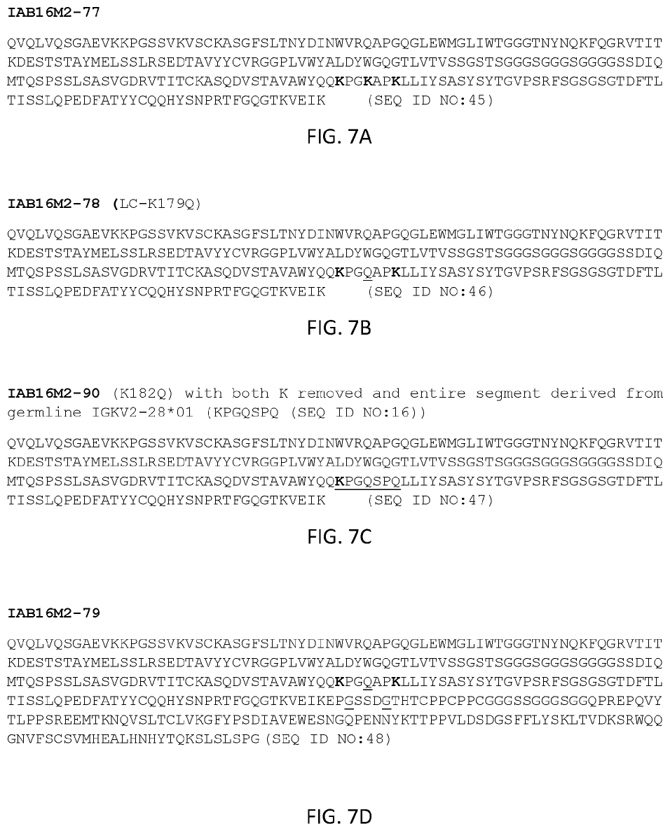

After conjugation with Df and labeling with ⁸⁹Zr, the labeled minibodies were administered to mice. The average radioactive uptakes in the liver and kidney were measured at 24 hours after administration as a percentage of injected dose per gram (% ID/g), and the results are shown in FIG. 6. The variant minibodies showed reduced accumulation in the kidneys and increased accumulation in the liver compared to the original minibody. The reduced accumulation in the kidneys is expected to reduce renal radiation toxicity. It may also allow the use of higher radiation dose in the context of radiotherapy.

### Example 2

This non-limiting example shows enhancing biodistribution of a minibody by mutating positively charged amino acids in a cluster of positively charged amino acids in the FR2 of the light chain variable region.

IAB16M2-77 is an anti-human-FAP minibody which was engineered through humanization of an antibody from a mouse hybridoma clone. This original antibody was humanized based on germlines: IGHV1-69*01/IGKV1-39*01. The germline IGKV1-39*01 is a commonly used light-chain germline sequence.

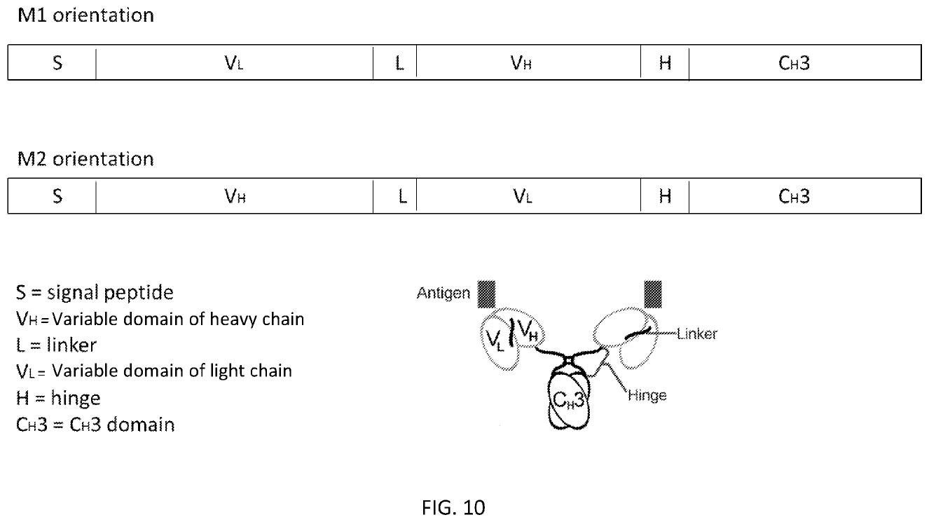

A positively charged patch on germline IGKV1-39*01 was identified in the framework region (FR) 2 of the light chain variable region (V_{L}), as shown in the minibody scFv amino acid sequence in FIG. 7A. The surface patch maps to sequence KPGKAPK (SEQ ID NO:5) which contains 3 charged residues (Lys —highlighted in bold in FIG. 7A) in a cluster (corresponding to K45, K48, and K51 by IMGT numbering). Mutation K48Q on the framework 2 of this germline sequence (underlined in FIG. 7B) reduced kidney uptake of ⁸⁹Zr radiolabeled minibodies at 24 hours after administration (FIG. 8). Distribution to the liver and spleen remained relatively unchanged. These results are consistent with the positively charged patch formed by the cluster of lysines being responsible for accumulating this minibody in the kidney.

### Example 3

This non-limiting example shows substitution of a segment of framework region 2 of one light chain germline sequence with a corresponding segment from another germline sequence to disrupt a cluster of positively charged residues.

As shown in Table 3.1, several human Kappa and Lambda light chain germline sequences have the pattern of three positively charged residues at framework region 2 (either K or R), including germline IGKV1-39*01. An analysis of the isopotential electrostatic surfaces of minibodies having the pattern of positively charged residues at framework region 2 in the light chain variable region using an Adaptive Poisson-Boltzmann Solver revealed a patch of positive charges formed by the positively charged residues whose center of masses are within a linear distance of about 11 angstroms of each other.

TABLE 3.1

| Germline | Sequence | SEQ ID NO |
|---|---|---|
| IGKV1-39*01 | WYQQKPGKAPKLLIY | 21 |
| IGKV1-33*01 | WYQQKPGKAPKLLIY | 21 |
| IGKV3-11*01 | WYQQKPGQAPRLLIY | 20 |
| IGKV3-20*01 | WYQQKPGQAPRLLIY | 20 |
| IGKV4-1*01 | WYQQKPGQPPKLLIY | 22 |
| IGKV1-NL*01 | WYQQKPGKAPKLLLY | 23 |
| IGKV1-27*01 | WYQQKPGKVPKLLIY | 24 |
| IGKV1-12*01 | WYQQKPGKAPKLLIY | 21 |
| IGKV2-29*02 | WYLQKPGQSPQLLIY | 25 |
| IGKV1-5*03 | WYQQKPGKAPKLLIY | 21 |
| IGKV1-16*01 | WFQQKPGKAPKSLIY | 26 |
| IGLV2-14*03 | WYQQHPGKAPKLMIY | 27 |
| IGKV3-15*01 | WYQQKPGQAPRLLIY | 20 |
| IGKV6-21*01 | WYQQKPDQSPKLLIKY | 28 |
| IGKV1-5*01 | WYQQKPGKAPKLLIY | 21 |
| IGKV1-51*01 | WYQQLPGTAPKLLIY | 29 |
| IGKV2D-29*02 | WYLQKPGQSPQLLIY | 25 |
| IGLV3-1*01 | WYQQKPGQSPVLVIY | 30 |
| IGKV3D-7*01 | WYQQKPGQAPRLLIY | 20 |
| IGKV1D-16*01 | WYQQKPEKAPKSLIY | 31 |
| IGKV1-13*02 | WYQQKPGKAPKLLIY | 21 |
| IGKV3-19*01 | WYQQKPGQAPVLVIY | 32 |
| IGKV1D-13*01 | WYQQKPGKAPKLLIY | 21 |
| IGKV1-9*01 | WYQQKPGKAPKLLIY | 21 |
| IGKV2-28*01 | WYLQKPGQSPQLLIY | 25 |
| IGLV6-57*01 | WYQQRPGSSPTTVIY | 33 |
| IGKV2-14*01 | WYQQHPGKAPKLMIY | 27 |
| IGKV1-40*01 | WYQQLPGTAPKLLIY | 29 |
| IGKV2-30*01 | WFQQRPGQSPRRLIY | 34 |
| IGKV1-17*01 | WYQQKPGKAPKRLIY | 35 |
| IGKV3D-11*01 | WYQQKPGQAPRLLIY | 20 |
| IGKV7-3*01 | WYQQKPGQPPKLLIY | 22 |
| IGLV7-43*01 | WFQQKPGQAPRALIY | 36 |
| IGLV3-21*02 | WYQQKPGQAPVLVVY | 37 |
| IGKV2-30*02 | WFQQRPGQSPRRLIY | 34 |
| IGLV3-21*03 | WYQQKPGQAPVLVVY | 37 |

As shown in FIG. 7C, KPGKAPK (SEQ ID NO:5) in germline IGKV1-39*01 was replaced with KPGQSPQ (SEQ ID NO: 16) from IGKV2-28*01. Germline IGKV1-39*01 is modified by substituting a segment of framework region 2 (FR2) (KPGKAPK (SEQ ID NO: 5)) with a similar sequence from germline sequences that do not contain this charge pattern, such as framework region 2 (FR2) from IGKV3-20*01 (KPGQAPR (SEQ ID NO:6)) or IGKV2-28*01 (KPGQSPQ (SEQ ID NO:16)).

A minibody that includes the variant V_{L} (having the modified FR2) is produced and radiolabeled. The radiolabeled minibody is administered to mice. 24 hours after administration, the animal is imaged and the distribution of the minibody in the animals is determined. The minibody having the variant V_{L} shows reduced accumulation in the kidneys compared to the original minibody.

### Example 4

This non-limiting example shows enhancing biodistribution of a cys-diabody by mutating positively charged amino acids in a cluster of positively charged amino acids in the FR2 of the light chain variable region.

A cys-diabody is constructed by fusing a humanized heavy chain variable region (V_{H}) antibody to the C-terminus of a humanized light chain variable region (V_{L}) of the antibody via a linker to form an scFv fragment, and adding an extension sequence that mediates dimerization to the C-terminus.

The humanized V_{L} uses the germline sequence of IGVK3-15*01. The V_{L} has a framework region 2 (FR2) sequence (WYQQKPGQAPRLLI (SEQ ID NO:20) (according to North)) that includes a cluster of two basic amino acids (lysine and arginine) at positions 45 and 51 (IMGT numbering). Molecular modeling of the surface charges using an Adaptive Poisson-Boltzmann Solver identifies a localized patch of surface-exposed positive charges by the clustering of these two basic amino acids. The localized positively charged patch may contribute to accumulation of cys-diabodies in the kidney.

R51 is substituted with a glutamine (Q). After conjugation with Df and labeling with ⁸⁹Zr, the labeled cys-diabodies are administered to mice. The average radioactive uptakes in the liver and kidney is measured at 24 hr post injection of the radiolabeled cys-diabody. The variant cys-diabodies show reduced accumulation in the kidneys and increased accumulation in the liver compared to the original cys-diabody.

### Example 5

This non-limiting example shows enhancing biodistribution of a minibody by mutating positively charged amino acids in a cluster of positively charged amino acids in the hinge region.

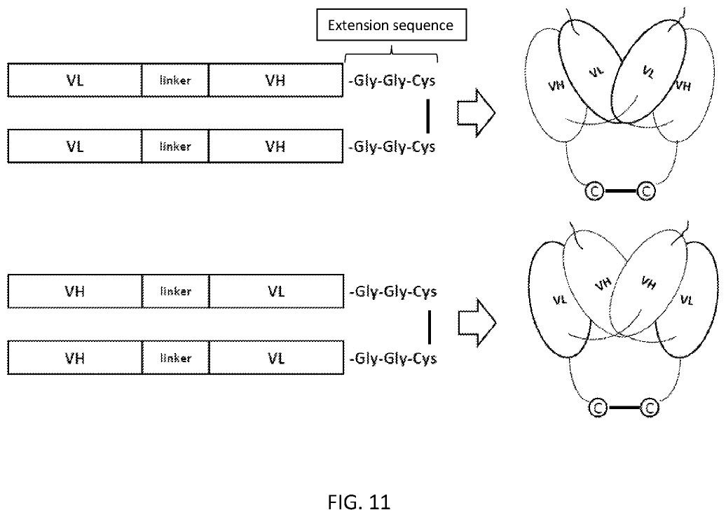

A minibody (JAB16M2-78) was constructed by fusing a humanized heavy chain variable region (V_{H}) antibody to the C-terminus of a humanized light chain variable region (V_{L}) of the antibody via a linker to form an scFv fragment, and fusing the scFv fragment to a C_{H}3 domain via a hinge region. The hinge region included an upper hinge sequence EPKSSDKTHT (SEQ ID NO:38) that includes a cluster of two lysines. The lysine residues in the cluster were substituted with a glycine (G) to generate a variant minibody, IAB16M2-79 (FIG. 7D), having the upper hinge sequence EPGSSDGTHT (SEQ ID NO:39). After conjugation with Df and labeling with ⁸⁹Zr, the labeled minibodies were administered to mice. The average radioactive uptake in the liver and kidney were measured at 24 hr post injection of the radiolabeled minibody. The variant minibodies showed reduced accumulation in the kidneys compared to the original minibody (FIG. 9).

### Example 6

This non-limiting example shows radioimmunotherapy treatment of a subject using an antibody of the present disclosure.

A subject in need of cancer treatment is identified. A variant minibody that specifically binds a target in the tumor and having the sequence KPGQAPQ (SEQ ID NO: 14) in the light chain variable region (V_{L}) framework region 2 (FR2) is labeled with a radionuclide. The radiolabeled minibody is administered to the subject. The radiation dose delivered to the subject is greater than the dose that would have been delivered using an original minibody having the sequence KPGQAPR (SEQ ID NO:6) in the V_{L} FR2, while renal toxicity remains comparable.

### Example 7

This non-limiting example shows treatment of a subject using an antibody of the present disclosure.

A subject in need of liver cancer treatment is identified. A variant minibody that specifically binds a target in the tumor and having the sequence KPGQAPQ (SEQ ID NO:4) in the light chain variable region (V_{L}) framework region 2 (FR2) is conjugated with a cytotoxic agent. The conjugated minibody is administered to the subject.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure has been presented in the context of certain embodiments and examples, those skilled in the art will understand that the present disclosure extends beyond the specifically disclosed embodiments to other alternative embodiments and/or uses of the disclosure and obvious modifications and equivalents thereof. In addition, while several variations of the present disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the present disclosure. It should be understood that various features and aspects of the disclosed embodiments can be combined with, or substituted for, one another in order to form varying modes or embodiments of the present disclosure. Thus, it is intended that the scope of the present disclosure herein presented should not be limited by the particular disclosed embodiments described above.

It should be understood, however, that this detailed description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the disclosures herein described.

What is claimed is:

1. A variant antibody comprising at least one disrupted cluster of surface-exposed positively charged amino acids, wherein the variant antibody varies from an original antibody comprising an original cluster comprising at least two surface-exposed, positively charged amino acids within about 30 angstroms of each other by having a substitution of at least one of the surface-exposed positively charged amino acids of the original cluster with a negatively charged or non-charged amino acid.

2. The variant antibody of claim 1, wherein the at least two surface-exposed, positively charged amino acids of the original cluster are within about 15 angstroms of each other.

3. The variant antibody of claim 1 or 2, wherein the variant antibody has at least about 10% less total positive charge attributed to the cluster compared to the original antibody due to disruption of the cluster.

4. The variant antibody of any one of the preceding claims, wherein the variant antibody varies from the original antibody by up to 3 amino acid substitutions that disrupt the cluster.

5. The variant antibody of any one of the preceding claims, wherein the substitution of at least one of the surface-exposed positively charged amino acids of the original cluster is in a light chain variable region (V_{L}) framework region (FR) of the original antibody.

6. The variant antibody of claim 5, wherein the substitution of at least one of the surface-exposed positively charged amino acids of the original cluster is in a V_{L} FR2 of the original antibody.

7. The variant antibody of any one of the preceding claims, wherein the original antibody comprises a polypeptide comprising an original sequence of X₁X₂X₃X₄X₅X₆X₇ (SEQ ID NO:2), wherein X₁ is a positively charged amino acid, wherein X₂, X₃, X₅, and X₆ are each independently a negatively charged or a non-charged amino acid, and wherein X₄ and X₇ are each independently any amino acid with the proviso that at least one is a positively charged amino acid, wherein the original sequence is outside of any CDR of the original antibody, wherein the at least one of X₄ and X₇ that is a positively charged amino acid is surface exposed and is part of the original cluster.

8. The variant antibody of claim 7, wherein the positive charges attributed to the original sequence is reduced by about 33% or more in the variant antibody.

9. The variant antibody of claim 8, wherein the positive charges attributed to the original sequence is reduced by about 60% or more in the variant antibody.

10. The variant antibody of any one of claims 7-9, wherein the substitution comprises substitution of the at least one of X₄ and X₇ with a negatively charged or non-charged amino acid.

11. The variant antibody of any one of claims 7-10, wherein the polypeptide comprises QQX₁X₂X₃X₄X₅X₆X₇ (SEQ ID NO:3) comprising the original sequence of X₁X₂X₃X₄X₅X₆X₇ (SEQ ID NO:2).

12. The variant antibody of any one of claims 7-11, wherein X₇ is a positively charged amino acid.

13. The variant antibody of any one of claims 7-12, wherein X₄ is a positively charged amino acid.

14. The variant antibody of any one of claims 7-13, wherein the positively charged amino acid is a lysine, arginine, or histidine.

15. The variant antibody of claim 14, wherein the positively charged amino acid is a lysine or arginine.

16. The variant antibody of any one of claims 7-15, wherein X₂ and/or X₆ is a proline.

17. The variant antibody of any one of claims 7-16, wherein X₃ is a glycine or glutamate.

18. The variant antibody of any one of claims 7-17, wherein X₅ is an alanine, valine, serine, or proline.

19. The variant antibody of any one of claims 7-18, wherein the original sequence is one of KX₂X₃KX₅ X₆K (SEQ ID NO:73), wherein X₂, X₃, X₅, and X₆ are each independently a negatively charged or a non-charged amino acid; KX₂X₃X₄X₅ X₆R (SEQ ID NO:74), wherein X₂, X₃, X₄, X₅, and X₆ are each independently a negatively charged or a non-charged amino acid; or KX₂X₃X₄X₅ X₆K (SEQ ID NO:75), wherein X₂, X₃, X₄, X₅, and X₆ are each independently a negatively charged or a non-charged amino acid.

20. The variant antibody of any one of claims 7-19, wherein the original sequence is one of: KPGKAPK (SEQ ID NO:5), KPGQAPR (SEQ ID NO:6), KPEKAPK (SEQ ID NO:7), KPGKVPK (SEQ ID NO:8), KPGQPPR (SEQ ID NO:9), KPGQSPR (SEQ ID NO:10), KPGLAPR (SEQ ID NO:11), or KPGQPPK (SEQ ID NO:12).

21. The variant antibody of claim 20, wherein the original sequence is KPGKAPK (SEQ ID NO:5) or KPGQAPR (SEQ ID NO:6).

22. The variant antibody of any one of claims 7-21, wherein the negatively charged or non-charged amino acid with which the at least one of the surface-exposed, positively charged amino acids of the cluster is substituted is a glutamine.

23. The variant antibody of any one of claims 7-22, wherein the original sequence is in a light chain variable region (V_{L}) framework region (FR) of the original antibody.

24. The variant antibody of claim 23, wherein the original sequence is in a V_{L} FR2 of the original antibody.

25. The variant antibody of any one of the preceding claims, wherein the variant antibody comprises at least one of KPGQAPK (SEQ ID NO: 13), KPGQAPQ (SEQ ID NO:14), KPGQSPQ (SEQ ID NO:16), and QQKPGQSPQ (SEQ ID NO:15) in a V_{L} FR2.

26. The variant antibody of any one of the preceding claims, wherein the variant antibody comprises V_{L} FR2 having an amino acid sequence of: WYQQKPGQAPQLLIY (SEQ ID NO:17), WYQQKPGQSPQLLIY (SEQ ID NO:18), or WYQQKPGQAPKLLIY (SEQ ID NO:19).

27. The method of any one of any one of the preceding claims, wherein the substitution of at least one of the surface-exposed positively charged amino acids of the original cluster is in a hinge region of the original antibody.

28. The method of claim 27, wherein the substitution of at least one of the surface-exposed positively charged amino acids of the original cluster is in an upper hinge region of the original antibody.

29. An antibody comprising a variant polypeptide that varies from an original polypeptide comprising an original sequence X₁X₂X₃X₄X₅X₆X₇ (SEQ ID NO:1), wherein the original sequence is outside of any CDR of the original polypeptide, wherein X₁, X₂, X₃, X₄, X₅, X₆, and X₇ are each independently any amino acid with the proviso that at least two are surface-exposed, positively charged amino acids that form a cluster of surface-exposed positively charged amino acids in the original polypeptide,
wherein the variant polypeptide varies from the original polypeptide by having a modification of at least one of the at least two surface exposed, positively charged amino acids of the original sequence that reduces at least 33% of positive charges attributed to the original sequence.

30. The antibody of claim 29, wherein the positive charges attributed to the original sequence is reduced by about 60% or more in the variant polypeptide.

31. The antibody of claim 29 or 30, wherein the number of positively charged amino acid in the original sequence is reduced by one or more in the variant polypeptide.

32. The antibody of any one of claims 29-31, wherein at least one of the at least two positively charged amino acids of the original sequence is substituted with a negatively charged or non-charged amino acid in the variant polypeptide.

33. The antibody of any one of claims 29-32, wherein at least X₄ and X₇ are each independently a positively charged amino acid.

34. The antibody of any one of claims 29-33, wherein the positively charged amino acid is a lysine, arginine, or histidine.

35. The antibody of any one of claims 29-34, wherein X₂, X₃, X₅, and X₆, are each independently a negatively charged or non-charged amino acid.

36. The antibody of any one of claims 29-35, wherein at least one of X₄ and X₇ is substituted with a negatively charged or non-charged amino acid to reduce the positive charges.

37. The antibody of claim 36, wherein the negatively charged or non-charged amino acid with which the at least one of X₄ and X₇ is substituted is a glutamine.

38. The antibody of any one of claims 29-37, wherein the variant polypeptide comprises an alanine or serine at a position corresponding to X₅ in the sequence of the original polypeptide.

39. The antibody of any one of claims 29-38, wherein the variant polypeptide comprises a light chain variable region (V_{L}) of the antibody.

40. The antibody of any one of claims 29-39, wherein the original sequence is within a V_{L} framework region (FR) of the original polypeptide.

41. The antibody of claim 40, wherein the original sequence is within a V_{L} FR2 of the original polypeptide.

42. The antibody of 40 or 41, wherein the original polypeptide is based on a first human germline sequence of a V_{L} having an original FR2 comprising the original sequence, and wherein the variant polypeptide varies from the original polypeptide by having a substitution of at least the original sequence with a corresponding, second sequence from a second FR2 from a second human germline sequence of a V_{L}, wherein the second FR2 comprises the second sequence corresponding to the original sequence and having at least 10% less positive charge attributed thereto compared to the original sequence.

43. A variant antibody comprising at least one disrupted cluster of positively charged amino acids, wherein the variant antibody varies from an original antibody comprising an original cluster comprising at least two positively charged amino acids within 12 residues of each other by having a substitution of at least one of the positively charged amino acids of the original cluster with a negatively charged or non-charged amino acid, wherein the original cluster is outside of any CDR of the original antibody.

44. The variant antibody of claim 43, wherein the original cluster is in a light chain variable region (V_{L}) FR of the original antibody.

45. The variant antibody of claim 44, wherein the original cluster is in a V_{L} FR2 of the original antibody

46. The variant antibody of claim 45, wherein the variant antibody comprises at least one of the following comprising the substitution of at least one of the positively charged amino acids: KPGQAPK (SEQ ID NO: 13), KPGQAPQ (SEQ ID NO: 14), and QQKPGQSPQ (SEQ ID NO:15).

47. The variant antibody of claim 45, wherein the variant antibody comprises V_{L}FR2 comprising an amino acid sequence of: WYQQKPGQAPQLLIY (SEQ ID NO:17), WYQQKPGQSPQLLIY (SEQ ID NO:18), or WYQQKPGQAPKLLIY (SEQ ID NO:19).

48. The variant antibody of claim 43, wherein the original cluster is in a hinge region of the original antibody.

49. The variant antibody of claim 48, wherein the original cluster is in an upper hinge region of the original antibody.

50. The variant antibody of claim 48 or 49, wherein the original antibody comprises an upper hinge sequence of EPKSSDKTHT (SEQ ID NO:38).

51. The antibody of any one of claims 48-50, wherein the variant antibody comprises an upper hinge sequence of EPGSSDGTHT (SEQ ID NO:39).

52. The antibody of any one of the preceding claims, wherein the antibody comprises an antigen-binding fragment.

53. The antibody of any one of the preceding claims, wherein the molecular weight of the antibody is in a range of about 15 kDa to about 110 kDa.

54. The antibody of any one of the preceding claims, wherein the antibody is a minibody, a cys-diabody, a scFv, a scFv-Fc, or a nanobody-Fc.

55. The antibody of any one of the preceding claims, wherein the molecular weight of the antibody is in a range of about 10 kDa to about 20 kDa.

56. The antibody of claim 55, wherein the antibody is a nanobody.

57. The antibody of any one of claims 1-54, wherein the molecular weight of the antibody is greater than 90 kDa.

58. The antibody of claim 57, wherein the antibody is an scFv-Fc.

59. A minibody or cys-diabody comprising a variant polypeptide that varies from an original polypeptide comprising a light chain variable region (V_{L}) FR2 comprising an original sequence X₁X₂X₃X₄X₅X₆X₇ (SEQ ID NO:1), wherein X₁, X₂, X₃, X₄, X₅, X₆, and X₇ are each independently any amino acid with the proviso that at least two are surface-exposed, positively charged amino acids that form a cluster of surface-exposed positively charged amino acids in the original polypeptide,
wherein the variant polypeptide varies from the original polypeptide by having a modification of at least one of the at least two surface exposed, positively charged amino acids of the original sequence that reduces at least 33% of positive charges attributed to the original sequence.

60. The minibody or cys-diabody of claim 59, wherein at least X₄ and X₇ are each independently a positively charged amino acid in the original polypeptide.

61. The minibody or cys-diabody of claim 59 or 60, wherein X₂, X₃, X₅, and X₆, are each independently a negatively charged or non-charged amino acid in the original polypeptide.

62. The minibody or cys-diabody of any one of claims 59-61, wherein at least one of X₄ and X₇ is substituted with a negatively charged or non-charged amino acid to reduce the positive charges.

63. A minibody or cys-diabody comprising a light chain variable region (V_{L}) comprising a FR2 sequence comprising X₁X₂X₃QAX₆X₇ (SEQ ID NO:4), wherein X₁ is a surface-exposed, positively charged amino acid, wherein X₂, X₃, and X₆, are each independently any negatively charged or non-charged amino acid, and wherein X₇ is either glutamine or lysine.

64. The minibody or cys-diabody of any one of claims 59-63, wherein X₁ is a lysine.

65. The minibody or cys-diabody of any one of claims 59-64, wherein X₂ and/or X₆ is a proline.

66. The minibody or cys-diabody of any one of claims 59-65, wherein X₃ is a glycine or glutamate.

67. The minibody or cys-diabody of claim 63, wherein the FR2 sequence comprises KPGQAPK (SEQ ID NO: 13) or KPGQAPQ (SEQ ID NO: 14).

68. A minibody or cys-diabody comprising a light chain variable region (V_{L}) comprising (by IMGT numbering):
A49 and either:
(i) Q51; or
(ii) Q48 and K51.

69. The minibody or cys-diabody of claim 68, wherein the V_{L} further comprises K45 (IMGT numbering).

70. A minibody comprising an upper hinge sequence of EPGSSDGTHT (SEQ ID NO:39).

71. The antibody, minibody, or cys-diabody of any one of the preceding claims, wherein the antibody, minibody, or cys-diabody specifically binds to DLL3, FAP, CD8, CD4, CD3, IFNγ, integrin αVβ6, FOLRα, or PSMA.

72. The antibody, minibody, or cys-diabody of any one of the preceding claims, wherein the antibody, minibody, or cys-diabody comprises a light chain variable region comprising 3 LCDR sequences in any one of the sequences in FIGS. 5B, 5C, 7B-7D, and a heavy chain variable region (V_{H}) comprising 3 HCDR sequences in any one of the sequences in FIGS. 5B, 5C, 7B-7D.

73. The antibody, minibody, or cys-diabody of any one of the preceding claims, further comprising a detectable label.

74. The antibody, minibody, or cys-diabody of claim 73, wherein the antibody, minibody, or cys-diabody is labeled with a radionuclide or an organic dye.

75. The antibody, minibody, or cys-diabody of any one of claims 1-72, further comprising a therapeutic agent.

76. The antibody, minibody, or cys-diabody of claim 75, wherein the therapeutic agent is a cytotoxic agent.

77. The antibody, minibody, or cys-diabody of claim 75, wherein the therapeutic agent is a radionuclide.

78. The antibody, minibody, or cys-diabody of claim 75, wherein the radionuclide is selected from among ²¹²Pb, ¹⁴⁹Tb and ¹⁶¹Tb.

79. A composition comprising:
an antibody, minibody or cys-diabody of any one of the preceding claims; and
a pharmaceutically acceptable carrier.

80. A nucleic acid encoding the variant polypeptide of the antibody of any one of claims 29-42 or the minibody or cys-diabody of any one of claims 59-62.

81. A nucleic acid encoding the variant antibody of any one of claims 1-28 and 43-54, the antibody of any one of claims 29-42, or the minibody or cys-diabody of any one of claims 59-72.

82. A genetically engineered host cell comprising the nucleic acid of claim 80 or 81.

83. A method of enhancing biodistribution and/or pharmacokinetics of an antibody, comprising:
identifying an original antibody comprising a polypeptide comprising at least one cluster of surface-exposed positively charged amino acids, the cluster comprising at least two surface-exposed, positively charged amino acids within 30 angstroms of each other;
substituting at least one of the at least two surface-exposed, positively charged amino acids of the at least one cluster with a negatively charged or non-charged amino acid to thereby disrupt the at least one cluster,
whereby a variant antibody having enhanced biodistribution and/or pharmacokinetics compared to the original antibody is generated.

84. A method of enhancing biodistribution and/or pharmacokinetics of an antibody, comprising:
identifying an original antibody comprising a polypeptide comprising a cluster of at least two surface-exposed, positively charged amino acids within 12 residues of each other; and
substituting at least one of the at least two surface-exposed, positively charged amino acids of the cluster with a negatively charged or non-charged amino acid to disrupt the cluster, thereby generating a variant antibody having enhanced biodistribution and/or pharmacokinetics compared to the original antibody.

85. The method of claim 84, wherein the cluster comprises the at least two positively charged amino acids within 6 residues of each other.

86. The method of claim 84 or 85, wherein the polypeptide comprises an amino acid sequence of X₁X₂X₃X₄X₅X₆X₇ (SEQ ID NO: 1), wherein X₁, X₂, X₃, X₄, X₅, X₆, and X₇ are each independently any amino acid, wherein the at least two surface-exposed, positively charged amino acids comprise at least two of X₁, X₂, X₃, X₄, X₅, X₆, and X₇.

87. The method of claim 86, wherein the at least two surface-exposed, positively charged amino acids comprise at least X₁.

88. The method of claim 86 or 87, wherein the at least two surface-exposed, positively charged amino acids comprise at least X₄ and X₇.

89. The method of any one of claims 86-88, wherein X₂, X₃, X₅, and X₆, are each independently a negatively charged or non-charged amino acid residue in the original polypeptide.

90. The method of any one of claims 86-89, wherein at least one of X₄ and X₇ are substituted with a negatively charged or non-charged amino acid.

91. The method of any one of claims 83-90, wherein the at least two surface-exposed, positively charged amino acids comprise arginine, lysine, or histidine.

92. The method of any one of claims 83-91, wherein the negatively charged or non-charged amino acid with which the at least one of the at least two surface-exposed, positively charged amino acids is substituted comprises a polar amino acid.

93. The method of claim 92, wherein the negatively charged or non-charged amino acid with which the at least one of the at least two surface-exposed, positively charged amino acids is substituted is glutamine.

94. The method of any one of claims 83-93, wherein the positively charged amino acid and the negatively charged or non-charged amino acid have side chain lengths that are different by less than one or two carbons.

95. The method of any one of claims 83-94, comprising substituting up to 3 amino acids of the polypeptide to disrupt the at least one cluster.

96. The method of any one of claims 83-95, wherein the cluster is outside of any complementarity determining region (CDR) of the original antibody.

97. The method of any one of claims 83-96, wherein the cluster is in a light chain variable region (V_{L}) of the original antibody.

98. The method of any one of claims 83-97, wherein the at least two surface-exposed, positively charged amino acids are within a V_{L} framework region (FR) of the original antibody.

99. The method of any one of claims 83-98, wherein the at least two surface-exposed, positively charged amino acids are within a V_{L} FR2 of the original antibody.

100. The method of claim 98 or 99, wherein the polypeptide is based on a first human germline sequence of a V_{L} having an original FR2 comprising the at least two surface-exposed, positively charged amino acids are, and wherein the method comprises substituting the original FR2, or a portion thereof comprising the cluster, with a second FR2, or a corresponding portion thereof, from a second human germline sequence of a V_{L}, wherein the second FR2 comprises at least one fewer surface-exposed, positively charged amino acids compared to the cluster.

101. The method of claim 99 or 100, wherein the original sequence is one of KX₂X₃KX₅ X₆K (SEQ ID NO:73), wherein X₂, X₃, X₅, and X₆ are each independently a negatively charged or a non-charged amino acid; KX₂X₃X₄X₅ X₆R (SEQ ID NO:74), wherein X₂, X₃, X₄, X₅, and X₆ are each independently a negatively charged or a non-charged amino acid; or KX₂X₃X₄X₅ X₆K (SEQ ID NO:75), wherein X₂, X₃, X₄, X₅, and X₆ are each independently a negatively charged or a non-charged amino acid.

102. The method of claim 99 or 101, wherein the original polypeptide comprises one of: KPGKAPK (SEQ ID NO:5), KPGQAPR (SEQ ID NO:6), KPEKAPK (SEQ ID NO:7), KPGKVPK (SEQ ID NO:8), KPGQPPR (SEQ ID NO:9), KPGQSPR (SEQ ID NO:10), KPGLAPR (SEQ ID NO:11), or KPGQPPK (SEQ ID NO:12) in a V_{L} FR2.

103. The method of any one of claims 99-101, wherein the variant polypeptide comprises at least one of KPGQAPK (SEQ ID NO:13), KPGQAPQ (SEQ ID NO:14), KPGQSPQ (SEQ ID NO:16), and QQKPGQSPQ (SEQ ID NO:15) in a V_{L} FR2.

104. The method of any one of claims 99-101, wherein the variant polypeptide comprises at least one of: WYQQKPGQAPQLLIY (SEQ ID NO: 17), WYQQKPGQSPQLLIY (SEQ ID NO:18), or WYQQKPGQAPKLLIY (SEQ ID NO:19) in a V_{L} FR2.

105. The method of any one of claims 83-104, further comprising conjugating a chelating ligand to the variant antibody.

106. The method of any one of claims 83-105, further comprising labeling the antibody with a radionuclide.

107. The method of any one of claims 83-105, further comprising labeling the antibody with a detectable label.

108. A method of making a labeled antibody, comprising:
selecting a germline sequence for a light chain variable region (V_{L}) of an antibody, wherein the germline sequence comprises no cluster of at least two positively charged amino acids within 3 residues of each other in a framework region 2 (FR2) of the germline sequence;
isolating one or more target-specific antibodies among a population of antibodies comprising a V_{L} derived from the germline sequence and having variations in the germline sequence across the population, wherein the one or more target-specific antibodies comprises no cluster of at least two positively charged amino acid within 3 residues of each other in a V_{L}FR2 sequence; and
labeling the one or more target-specific antibodies.

109. The method of claim 108, wherein isolating the one or more target-specific antibodies comprises screening a population of antibodies derived from the germline sequence.

110. The method of claim 109, wherein the population of antibodies derived from the germline sequence are generated in a host organism genetically modified to express antibodies based only on the selected germline sequence.

111. The method of claim 109, wherein the population of antibodies derived from the germline sequence is comprised in a phage library of antibodies based only on the selected germline sequence.

112. The method of any one of claims 109-111, wherein the germline sequence is a human germline sequence.

113. The method of any one of claims 108-112, wherein the one or more target-specific antibodies are humanized.

114. The method of any one of claims 108-113, further comprising generating one or more target-specific minibodies or cys-diabodies from the one or more target-specific antibodies.

115. The method of any one of claims 108-114, comprising labeling the one or more target-specific antibodies with a radionuclide.

116. The method of any one of claims 108-115, wherein labeling the one or more target-specific antibodies comprises conjugating a chelating ligand to the one or more target-specific antibodies.

117. The method of any one of claims 83-116, wherein the antibody comprises an antigen-binding fragment.

118. The method of any one of claims 83-117, wherein the antibody is a minibody or a cys-diabody.

119. The method of any one of claims 83-118, wherein the antibody specifically binds to DLL3, FAP, CD8, CD4, CD3, IFNγ, integrin αVβ6, FOLRα, or PSMA.

120. The method of any one of claims 83-118, wherein the antibody or minibody comprises a light chain variable region (V_{L}) comprising 3 LCDR sequences of the 3 LCDRs of any one of the sequences in FIGS. 5A-5C and 7A-7D, and a heavy chain variable region (V_{H}) comprising the 3 HCDR sequences of 3 HCDRs of any one of the sequences in FIGS. 5A-5C and 7A-7D.

121. An antibody made by the method of any one of claims 83-120.

122. A method of treating a subject, comprising:
identifying a subject in need of treatment with the antibody, minibody, or cys-diabody of any one of claims 1-78; and
administering a therapeutically effective amount of the antibody or minibody, or the composition of claim 79 to the subject.

123. A method of treating a subject for a cancer, comprising:
identifying a subject in need of treatment for a cancer; and
administering to the subject a therapeutically effective amount of the antibody, minibody, or cys-diabody of any one of claims 1-78, or the composition of claim 79, to thereby treat the cancer.

124. A method of radiotherapy, comprising:
identifying a subject in need of radiotherapy; and
administering to the subject a therapeutically effective amount of the antibody, minibody, or cys-diabody of any one of claims 1-78, or the composition of claim 79,
wherein the antibody, minibody, or cys-diabody comprises a radionuclide.

125. A method of imaging a subject, comprising:
administering to a subject a composition comprising an effective amount of the antibody, minibody, or cys-diabody of any one of claims 1-78 or the composition of claim 79,
wherein the antibody, minibody, or cys-diabody is detectably labeled; and
imaging the subject to detect the labeled antibody, minibody, or cys-diabody in the subject.

126. Use of the antibody, minibody, or cys-diabody of any one of claims 1-78 or the composition of claim 79, for treatment of cancer in a subject in need thereof.

127. Use of the antibody, minibody, or cys-diabody of any one of claims 1-78 for preparation of a medicament for treatment of cancer in a subject in need thereof.


SEQUENCE LISTING (76 sequences)

SEQ ID NO: 1-4 - skipped (4 entries)

SEQ ID NO: 5 - protein, 7 aa, synthetic construct
KPGKAPK

SEQ ID NO: 6 - protein, 7 aa, synthetic construct
KPGQAPR

SEQ ID NO: 7 - protein, 7 aa, synthetic construct
KPEKAPK

SEQ ID NO: 8 - protein, 7 aa, synthetic construct
KPGKVPK

SEQ ID NO: 9 - protein, 7 aa, synthetic construct
KPGQPPR

SEQ ID NO: 10 - protein, 7 aa, synthetic construct
KPGQSPR

SEQ ID NO: 11 - protein, 7 aa, synthetic construct
KPGLAPR

SEQ ID NO: 12 - protein, 7 aa, synthetic construct
KPGQPPK

SEQ ID NO: 13 - protein, 7 aa, synthetic construct
KPGQAPK

SEQ ID NO: 14 - protein, 7 aa, synthetic construct
KPGQAPK

SEQ ID NO: 15 - protein, 9 aa, synthetic construct
QQKPGQSPQ

SEQ ID NO: 16 - protein, 7 aa, synthetic construct
KPGQSPQ

SEQ ID NO: 17 - protein, 15 aa, synthetic construct
WYQQKPGQAPQLLIY

SEQ ID NO: 18 - protein, 15 aa, synthetic construct
WYQQKPGQSPQLLIY

SEQ ID NO: 19 - protein, 15 aa, synthetic construct
WYQQKPGQAPKLLIY

SEQ ID NO: 20 - protein, 15 aa, synthetic construct
WYQQKPGQAPRLLIY

SEQ ID NO: 21 - protein, 15 aa, synthetic construct
WYQQKPGKAPKLLIY

SEQ ID NO: 22 - protein, 15 aa, synthetic construct
WYQQKPGQPPKLLIY

SEQ ID NO: 23 - protein, 15 aa, synthetic construct
WYQQKPGKAPKLLLY

SEQ ID NO: 24 - protein, 15 aa, synthetic construct
WYQQKPGKVPKLLIY

SEQ ID NO: 25 - protein, 15 aa, synthetic construct
WYLQKPGQSPQLLIY

SEQ ID NO: 26 - protein, 15 aa, synthetic construct
WFQQKPGKAPKSLIY

SEQ ID NO: 27 - protein, 15 aa, synthetic construct
WYQQHPGKAPKLMIY

SEQ ID NO: 28 - protein, 16 aa, synthetic construct
WYQQKPDQSPKLLIKY

SEQ ID NO: 29 - protein, 15 aa, synthetic construct
WYQQLPGTAPKLLIY

SEQ ID NO: 30 - protein, 15 aa, synthetic construct
WYQQKPGQSPVLVIY

SEQ ID NO: 31 - protein, 15 aa, synthetic construct
WYQQKPEKAPKSLIY

SEQ ID NO: 32 - protein, 15 aa, synthetic construct
WYQQKPGQAPVLVIY

SEQ ID NO: 33 - protein, 15 aa, synthetic construct
WYQQRPGSSPTTVIY

SEQ ID NO: 34 - protein, 15 aa, synthetic construct
WFQQRPGQSPRRLIY

SEQ ID NO: 35 - protein, 15 aa, synthetic construct
WYQQKPGKAPKRLIY

SEQ ID NO: 36 - protein, 15 aa, synthetic construct
WFQQKPGQAPRALIY

SEQ ID NO: 37 - protein, 15 aa, synthetic construct
WYQQKPGQAPVLVVY

SEQ ID NO: 38 - protein, 10 aa, synthetic construct
EPKSSDKTHT

SEQ ID NO: 39 - protein, 10 aa, synthetic construct
EPGSSDGTHT

SEQ ID NO: 40 - protein, 10 aa, synthetic construct
GGGSSGGGSG

SEQ ID NO: 41 - protein, 18 aa, synthetic construct
GSTSGGGSGGGSGGGGSS

SEQ ID NO: 42 - protein, 396 aa, synthetic construct
METDTLLLWVLLLWVPGSTGEIVMTQSPATLSVSPGERATLSCKASQSVSNDVVWYQQKPGQAPRLLIYDASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 396 aa, synthetic construct
METDTLLLWVLLLWVPGSTGEIVMTQSPATLSVSPGERATLSCKASQSVSNDVVWYQQKPGQAPQLLIYDASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 396 aa, synthetic construct
METDTLLLWVLLLWVPGSTGEIVMTQSPATLSVSPGERATLSCKASQSVSNDVVWYQQKPGQSPQLLIYDASNRYTGIPARFSGSGSGTEFTLTISSLQSEDFAVYYCQQDYTSPWTFGQGTKLEIKGSTSGGGSGGGSGGGGSSQVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 244 aa, synthetic construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGKAPKLLIYSASYSYTGVPSRFSGSGSGTDFTLTISSLQPEDFATYYCQQHYSNPRTFGQGTKVEIK

SEQ ID NO: 46 - protein, 244 aa, synthetic construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGQAPKLLIYSASYSYTGVPSRFSGSGSGTDFTLTISSLQPEDFATYYCQQHYSNPRTFGQGTKVEIK

SEQ ID NO: 47 - protein, 244 aa, synthetic construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGQSPQLLIYSASYSYTGVPSRFSGSGSGTDFTLTISSLQPEDFATYYCQQHYSNPRTFGQGTKVEIK

SEQ ID NO: 48 - protein, 377 aa, synthetic construct
QVQLVQSGAEVKKPGSSVKVSCKASGFSLTNYDINWVRQAPGQGLEWMGLIWTGGGTNYNQKFQGRVTITKDESTSTAYMELSSLRSEDTAVYYCVRGGPLVWYALDYWGQGTLVTVSSGSTSGGGSGGGSGGGGSSDIQMTQSPSSLSASVGDRVTITCKASQDVSTAVAWYQQKPGQAPKLLIYSASYSYTGVPSRFSGSGSGTDFTLTISSLQPEDFATYYCQQHYSNPRTFGQGTKVEIKEPGSSDGTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 52 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 53 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 54 - protein, 107 aa, synthetic construct
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 55 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNVFSCSVMHEALHNHYTQKSLSLSPGK

SEQ ID NO: 56 - protein, 107 aa, synthetic construct
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 57 - protein, 107 aa, synthetic construct
GQPREPQVYTLPPSREEMTKNQVSLTCLVKGFYPSDIAVEWESSGQPENNYNTTPPVLDSDGSFFLYSRLTVDKSRWQEGNVFSCSVMHEALHNRFTQKSLSLSPGK

SEQ ID NO: 58 - protein, 107 aa, synthetic construct
GQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 59 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNRFTQKSLSLSPGK

SEQ ID NO: 60 - protein, 107 aa, synthetic construct
GQPREPQVYTLPPSREEMTKNQVSLTCLVKGFYPSDIAVEWESSGQPENNYKTTPPMLDSDGSFFLYSKLTVDKSRWQEGNIFSCSVMHEALHNRFTQKSLSLSPGK

SEQ ID NO: 61 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNRYTQKSLSLSPGK

SEQ ID NO: 62 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNIFSCSVMHEALHNRYTQKSLSLSPGK

SEQ ID NO: 63 - protein, 107 aa, synthetic construct
GQPREPQVYTLPPSREEMTKNQVSLTCLVKGFYPSDIAMEWESSGQPENNYKTTPPVLDSDGSFFLYSKLTVDKSRWQQGNIFSCSVMHEALHNHYTQKSLSLSPGK

SEQ ID NO: 64 - protein, 107 aa, synthetic construct
GQPREPQVYTLPPSREEMTKNQVSLTCLVKGFYPSDIAVEWESNGQRENNYNTTPPMLDSDGSFFLYSKLTVDKSRWQQGNIFSCSVMHEALHNRYTQKSLSLSPGK

SEQ ID NO: 65 - protein, 107 aa, synthetic construct
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNHYTQKSLSLSPGK

SEQ ID NO: 66 - protein, 107 aa, synthetic construct
GQPREPQVYTLPPSREEMTKNQVSLTCLVKGFYPSDIAVEWESSGQPENNYKTTPPVLDSDGSFFLYSKLTVDKSRWQQGNIFSCSVMHEALHNHYTQKSLSLSPGK

SEQ ID NO: 67 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 107 aa, synthetic construct
GQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 69 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 14 aa, synthetic construct
WYQQKPGQAPRLLI

SEQ ID NO: 71 - protein, 17 aa, synthetic construct
EPGSSDGTHTCPPCPPC

SEQ ID NO: 72 - protein, 9 aa, synthetic construct
GGCPPCPPC

SEQ ID NO: 73-75 - skipped (3 entries)

SEQ ID NO: 76 - protein, 225 aa, synthetic construct
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK